SEQUENCE LISTING (3 sequences)

SEQ ID NO: 1 - protein, 12 aa, Homo sapiens
LSEPAELTDAVK

SEQ ID NO: 2 - protein, 12 aa, synthetic construct
  REGION 1..12: note = Internal peptide standard
LSEPAELTEAVK

SEQ ID NO: 3 - protein, 9 aa, Homo sapiens
  SITE 1: note = N is position 69 in human PSA protein
NKSVILLGR

(19) United States
(12) Patent Application Publication
(10) Pub. No.: US 2025/0116670 A1
(43) Pub. Date: Apr. 10, 2025

(54) METHODS FOR MANAGEMENT OF PROSTATE CANCER

(71) Applicant: Roche Diagnostics Operations, Inc., Indianapolis, IN (US)

(72) Inventors: Christa Cobbaert, Leiden (NL); Konstantin Kroeniger, Penzberg (DE); Guinevere Lageveen-Kammeijer, Leiden (NL); Jan Nouta, Leiden (NL); Vinzent Rolny, Penzberg (DE); Renee Ruhaak, Leiden (NL); Magdalena Swiatek-de Lange, Penzberg (DE); Gloria Tabares, Penzberg (DE); Yuri van der Burgt, Leiden (NL); Manfred Wuhrer, Leiden (NL)

(21) Appl. No.: 18/908,787

(22) Filed: Oct. 7, 2024

Related U.S. Application Data
(63) Continuation of application No. PCT/EP2023/059129, filed on Apr. 6, 2023.

(30) Foreign Application Priority Data
Apr. 8, 2022 (EP) 22167487.2

(51) Int. Cl. G01N 33/574; G01N 33/573

(52) U.S. Cl. CPC G01N 33/57434; G01N 33/573; G01N 2333/96433; G01N 2440/38

(57) ABSTRACT

The present invention relates to methods for assessing whether or not a patient has aggressive prostate cancer by determining the levels of particular glycoforms attached to prostate specific antigen (PSA) protein in a biofluid sample of a subject, and comparing the determined level or concentration to a reference. The methods are particularly useful for assessing subjects that have 2-10 ng/ml total PSA in the subject's serum.

### BACKGROUND TO THE INVENTION

Worldwide more than 1.4 million patients were diagnosed with prostate cancer (PCa) in 2020 (Sung et al., CA Cancer J. Clin. 71:209-249, 2021). The concentration of prostate-specific antigen (PSA) in serum is used as an early detection and prediction method of PCa; however, this test exhibits low sensitivity, specificity, and has poor predictive value (Wolf et al., Ca-Cancer J. Clin. 60:70-98, 2010). Initial studies indicated that the glycosylation of PSA could provide a promising lead for more specific PCa diagnosis. Recently, the Center for Proteomics and Metabolomics (CPM) at Leiden University Medical Center established a high-performance PSA Glycomics Assay (PGA) that allows a detailed glycosylation analysis including the differentiation of α2,6- and α2,3-sialylated isomers (FIG. 1; Kammeijer et al., Anal. Chem. 90:4414-4421, 2018). Those isomers have also being suggested to be potential biomarkers of aggressive types of cancer (Schultz et al., Cancer Metastasis Rev. 31:501-518, 2012). The PGA comprises a PSA affinity purification step, followed by tryptic digestion and mass spectrometry (MS) analysis by capillary electrophoresis-electrospray ionization (CE-ESI) (Kammeijer et al., Anal. Chem. 90:4414-4421, 2018),

The use of PSA as a biomarker has serious shortcomings: first, PSA is not a PCa-specific biomarker as its body fluid levels (commonly blood) do not efficiently distinguish between PCa and other prostatic diseases, such as benign prostatic hyperplasia (BPH) or prostatitis (Hudson et al., J Urol. 151:1291-1291, 1989). Furthermore, serum levels of PSA are influenced by several factors as age, comorbidities, ejaculation, catheterization and medications (Hatekeyama et al., Int J Clin Oncol, 22:214-221, 2017). PSA-differentiation between PCa and prostatic benign diseases is especially inefficient in the so-called “grey area” ranging from 2-10 ng/ml (Barry, M. J., N Engl J Med. 360(13):1351-4, 2009) and in discriminating between indolent and aggressive PCa (Lamy et al., Eur Urol Focus. 4(6):790-803, 2018).

Screening for PCa with PSA has led to a reduction in advanced disease and disease-specific mortality. However, a trade-off is overdiagnosis of cases that would not have caused clinical consequences during a man's lifetime if left untreated. In turn, overdiagnosis has led to overtreatment with significant risks as side effects from biopsy or negative outcomes from treatment (Loeb et al., Eur Urol, 65(6):1046-1055, 2014). According to www.uspreventiveservicestaskforce.org (Final Recommendation Statement: Screening for Prostate Cancer and Final Evidence Review: Screening for Prostate Cancer. U.S. Preventive Services Task Force. May 2018. www.uspreventiveservicestaskforce.org), 20-50% of positively diagnosed men are having an indolent, non-threatening form of PCa. Those data clearly demonstrate a major problem of overdiagnosis as well as the need for improved, more specific tools for PCa diagnosis, and assessment of cancer aggressiveness.

The single N-linked glycosylation site (N69) of PSA is occupied by a very heterogeneous group of glycans, resulting in various so-called glycoforms with the same protein backbone. Previous studies speculated that detailed analysis of PSA glycoforms could offer a more efficient PCa diagnosis(Vermassen et al (supra); Jankovic et al., Clin. Biochem. 38:58-65, 2005; Peracaula et al., Glycobiology. 13:457-470, 2003); Saldova et al. Glycobiology. 21:195-205, 2011). Follow-up studies on the molecular features of PSA glycosylation (such as antenna modification and core fucosylation) also suggested an improved PCa diagnosis by combining the traditional PSA test with specific N-glycan features (Kyselova et al. J. Proteome Res. 6:1822-1832, 2007; Vermassen et al. Electrophoresis. 35:1017-1024, 2014; Yoneyama et al., Biochem. Biophys. Res. Commun. 448:390-396, 2014), however the size of the tested cohorts was limited and there is still a high need to identify new glycol structures. Furthermore, there is a need to identify biomarkers for assessing PCa aggressiveness.

Detection of PSA glycoforms is currently performed using lectin- or mass spectrometry-based methods (Llop et al., Theranostics. 6(8):1190-204, 2016; Kuzmanov et al., BMC Med. 11:31, 2013). The limitation of lectin application is reactivity only toward sugar residues and not to the protein backbone. Additionally, specificity of lectins to differentiate between related glycan structures is low, and their reactivity is mostly based on avidity and not affinity. On the other hand, mass spectrometry-based require more purification steps, especially in the case of complex matrices (e.g. serum or plasma).

### SUMMARY OF THE INVENTION

The present inventors have demonstrated that prostate-specific antigen (PSA), which is secreted from prostate cells into biological fluids such as blood and urine, is variously glycosylated, and have surprisingly found that levels of particular PSA glycoforms increase whilst levels of others decrease in subjects with aggressive forms of PCa. The present invention relies on these changes in levels of one or more particular PSA glycoform(s) to detect if a subject has or is likely to have an aggressive form of PCa or not. The methods of the invention can therefore be used to determine whether a subject with or suspected of having a prostatic disease (which may be any form of PCa or a non-cancerous prostatic disorder such as prostatitis or BPH) likely has indolent disease or aggressive PCa. This characterisation can then be used to inform the next therapeutic or analytic intervention. For example, an individual determined to likely have aggressive PCa can be selected for biopsy and/or therapeutic treatment. An individual determined that is likely to not have aggressive PCa can be selected for “watchful waiting”, i.e. regular monitoring but no (direct) intervention.

According to a first aspect of the invention, provided is a method that aids in determining whether or not a subject has an aggressive form of prostate cancer, comprising:
(a) determining the level of at least one mono-antennary PSA glycoform and the level of at least one di-antennary PSA glycoform in a biofluid sample from the subject;
(b) calculating an aggressiveness score for the subject using the levels determined in (a);
(c) comparing the subject's aggressiveness score in (b) to a reference value for said aggressiveness score; and
(d) using the comparison in (c) to aid in determining whether the subject has an aggressive form of prostate cancer or not.

According to a second aspect of the invention, provided is a method for determining whether or not a subject has an aggressive form of prostate cancer, comprising:
(i) isolating PSA protein from other proteins in a biofluid sample from the subject;
(ii) treating the isolated PSA protein with a protease;
(iii) determining the level of each of two or more N₆₉ glycopeptide forms in the sample, wherein at least one of the N₆₉ glycopeptide forms is a mono-antennary glycoform and at least one of the N₆₉ glycopeptide forms is a di-antennary glycoform;
(iv) determining an aggressiveness score for the subject using the levels of each N₆₉ glycopeptide forms in (iii);
(v) comparing the aggressiveness score in (iv) to a reference value for said aggressiveness score; and
(vi) based on the comparison in step (v) determining whether the subject has an aggressive form of PCa or not.

In particular embodiments, the determined levels in step (iii) are normalised levels. In particular embodiments, PSA protein is isolated from a biofluid sample from the subject using a binding protein (such as an antibody) specific for PSA protein.

In a particular embodiment, the level of a PSA glycoform corresponds to a normalised amount of the PSA glycoform (e.g. N₆₉ glycopeptide) relative to the sum of amounts of all measured PSA glycoforms (e.g. all glycoforms as shown in Table 1A).

In a particular embodiment, the level of a PSA glycoform corresponds to a normalised signal (e.g. area under the curve) correlating with the amount of the PSA glycoform (e.g. Nee glycopeptide) which is normalized to the signal of all measured PSA glycoforms (e.g. all glycoforms as shown in Table 1A).In a particular embodiment, the PSA protein is isolated/separated from other components in the biofluid sample, such as other proteins, using a binding protein (such as an antibody) specific for PSA protein.

It will be appreciated that the PSA protein isolated may contain numerous distinct proteoglycan species (PSA glycoforms).The protease serves to digests the PSA protein into smaller peptide forms so that the various proteoglycan species are attached to a smaller peptide. Suitable protease is trypsin which will produce the N₆₉K dipeptide proteoglycan population. Another suitable protease is Arg-C (clostripain) which will generate the NsgKSVILLGR (SEQ ID NO: 3) proteoglycan population.

In particular embodiments, the aggressiveness score is determined as for the first aspect of the invention. In particular embodiments, the reference aggressiveness score was determined as for the first aspect of the invention.

The first and second aspects of the invention involve measurements of the levels of at least two PSA glycoforms. However, as can be seen from Table 6, the levels of three PSA glycoforms (compounds 21, 2 and 9) are individually capable of discriminating aggressive from indolent PCa and BPH better (higher area under the curve (AUC) in receiver operating characteristic (ROC) analysis) than free PSA. The level of each of these three PSA glycoforms can therefore be used individually in a method to aid in determining whether the subject has aggressive PCa or not.

Thus, according to a third aspect of the invention there is provided; a method to aid in determining whether or not a subject has an aggressive form of prostate cancer, comprising:
(a) determining the level of a PSA glycoforms selected from the group consisting of: H4N5F1S_{2,3}1S_{2,6}1; H4N3S_{2,6}1; and H5N4F1S_{2,6}1 in a biofluid sample from the subject;
(b) comparing the value for the level of the PSA glycoform in (a) to a reference value for said PSA glycoform; or comparing an aggressiveness score taking into account the level said PSA glycoform in (a) to a reference value for said aggressiveness score; and
(c) using the comparison in (b) to aid in determining whether the subject has an aggressive form of prostate cancer or not.

According to a fourth aspect of the invention there is provided; a computer-implemented method to aid in determining whether a subject has an aggressive form of prostate cancer or not, comprising the steps of:
(a) receiving a value for the level of a first mono-antennary PSA glycoform in a biofluid sample of the subject;
(b) receiving a value for the level of a first di-antennary PSA glycoform in the biofluid sample of the subject;
(c) calculating an aggressiveness score for the subject based on the levels received in (a) and (b);
(d) comparing the subject's aggressiveness score in (c) to a reference value of said aggressiveness score; and
(e) using the comparison in (d) to aid in determining whether the subject has an aggressive form of prostate cancer or not.

In particular embodiments, the first mono-antennary PSA glycoform of step (a) is selected from compound 2, 3, 4 or 5 (as shown in Tables 1A and B).

In particular embodiments, the first di-antennary PSA glycoform of step (b) is selected from compound 7, 9, 10, 11, 13, 16, 18, 21, 24 or 25, (as shown in Tables 1A and B).

In a particular embodiment, the aggressiveness score in step (c) is calculated from a combination of the levels received in steps (a) and (b). In a particular embodiment, the aggressiveness score in step (c) is calculated from importing the levels received in steps (a) and (b) into an equation (e.g. a weighted calculation matrix, a ratio etc.).

The values received in (a) and (b) can be obtained from the PSA glycoform determined in the first aspect of the invention. As appropriate, the embodiments applicable to the first aspect of the invention can be applied to this fourth aspect of the invention.

According to a fifth aspect of the invention that is provided; a computer-implemented method to aid in determining whether a subject has an aggressive form of PCa or not, comprising the steps of:
(a) receiving a value for the level of a PSA glycoform selected from the group consisting of H4N5F1S_{2,3}1S_{2,6}1; H4N3S_{2,6}1; and H5N4F1S_{2,6}1 in a biofluid sample of the subject;
(b) comparing the value for the level of the PSA glycoform received in (a) to a reference value for said PSA glycoform; or comparing an aggressiveness score taking into account the level of the PSA glycoform received in (a) to a reference value for said aggressiveness score; and
(c) using the comparison in (b) to aid in determining whether or not the subject has an aggressive form of prostate cancer.

In a particular embodiment of the fifth aspect of the invention, provided is a computer-implemented method to aid in determining whether a subject has an aggressive form of prostate cancer or not, comprising the steps of:
(a) receiving a value for the level of a PSA glycoform selected from the group consisting of H4N5F1 S_{2,3}1S_{2,6}1; H4N3S_{2,6}1; and H5N4F1S_{2,6}1 in a biofluid sample of the subject;
(b) comparing the value for the level of the PSA glycoform received in (a) to a reference value for said PSA glycoform; and
(c) using the comparison in (b) to aid in determining whether or not the subject has an aggressive form of prostate cancer.

The value received in (a) can be obtained from the PSA glycoform determined in the third aspect of the invention. As appropriate, the embodiments applicable to the third aspect of the invention can be applied to this fifth aspect of the invention.

Suitably, the mono-antennary PSA glycoform for use in the methods according to the first, second, fourth and fifth aspects of the invention are selected from one or more of compounds 2, 3, 4 and 5, as identified in Table 1A and B.

Suitably, the di-antennary PSA glycoform for use in the methods according to the first, second, fourth and fifth aspects of the invention are selected from one or more of compounds 7, 9, 10, 11, 13, 16, 18, 21, 24 and 25, as identified in Table 1A and B.

The method of the first, second, third, fourth and fifth aspects of the invention can be used to aid in determining whether a subject has an aggressive form of PCa or to aid in determining whether a subject does not have an aggressive form of PCa, such as an indolent prostatic disease. Suitably, the method of the first, second, third, fourth or fifth aspects of the invention can be used to aid in determining whether the subject has an indolent prostatic disease, such as BPH.

According to a sixth aspect of the invention there is provided a kit for use (or use of a kit) in or with the method according to the first, second, third, fourth or fifth aspects of the invention, comprising a prostate cancer aggressiveness score or scoring system (e.g. a determining system with PSA glycoform threshold values that are indicative of aggressive PCa). Optionally, the kit also comprises a PSA protein binding agent, such as a monoclonal antibody. Such a PSA protein binding agent may be used for isolating or purifying PSA protein. In embodiments, the kit may comprise a protease. The protease can be used to generate glycopeptides of PSA that can be measured via mass spectrometry. In embodiments, the kit may be a kit for mass spectrometry detection of PSA glycoforms.

### DETAILED DESCRIPTION OF THE INVENTION

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The disclosed methods may be understood more readily by reference to the following detailed description which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

The methods of the present disclosure will employ, unless otherwise indicated, conventional techniques of molecular biology (including recombinant techniques), microbiology, cell biology, and biochemistry, which are within the skill of the art. Exemplary techniques are explained fully in the literature, such as, “Molecular Cloning: A Laboratory Manual”, 2^{nd} edition (Sambrook et al., Molecular Cloning: A Laboratory Manual, New York: Cold Spring Harbor Press, 1989); “Current Protocols in Molecular Biology” (F. M. Ausubel et al., eds., Current Protocols of Molecular Biology, John Wiley and Sons (1987); and “PCR: The Polymerase Chain Reaction”, (Mullis et al., eds., Birhäuser, Boston, 1994).

It is to be appreciated that certain features of the disclosed methods, which are for clarity, described herein in the context of separate embodiments, may also be provided in combination in a single embodiment. Conversely, various features of the disclosed methods that are, for brevity, described in the context of a single embodiments, may also be provided separately or in any sub-combination.

In describing the present invention, the following terms will be employed, and are intended to be defined as indicated below.

As used in this specification and the appended claims, the singular forms “a”, “an”, and “the” include plural referents, unless the content clearly dictates otherwise. As used herein, the term “species” as in “PSA-glycoform species” can be singular or plural depending on the context.

The use of the alternative (e.g., “or”) should be understood to mean either one, both, or any combination thereof of the alternatives.

The term “and/or” should be understood to mean either one, or both of the alternatives.

As used herein and unless stated otherwise, it is to be understood that the term “about” is used synonymously with the term “approximately”. Illustratively and unless stated otherwise, the use of the term “about” when used in conjunction with a stated numerical value or range denotes somewhat more or somewhat less than the stated value or range, to within a range of ±15% of that stated, ±10% of that stated, 5% of that stated, or conveniently ±2% of that stated. Such values are thus encompassed by the scope of the claims reciting the terms “about” or “approximately”.

The word “comprise”, and variations such as “comprises” and “comprising”, will be understood to imply the inclusion of a stated integer or step or group of integers or steps but not the exclusion of any other integer or step or group of integers or steps.

The term “analyte” refers to the molecule (e.g. PSA glycoform or protein) being detected and measured.

The term “level” or “amount” as used herein encompasses the absolute level or amount of a biomarker (e.g. a PSA glycoform) as referred to herein, a normalised amount, the relative amount or concentration of the said biomarker (e.g. a normalised signal (e.g. area under the curve) of a biomarker (e.g. PSA glycoform) to the sum signal of all integrated or measured PSA glycoforms (e.g. if 5 PSA glycoforms are measured the level could be PSA glycoform 1 amount or area under curve divided by the equivalent measure of PSA glycoforms 1+2+3+4+5; see also Table 1A)), as well as any value or parameter which correlates thereto or can be derived therefrom. Such values or parameters may comprise the integrated area values or intensity signal from all specific physical or chemical properties obtained from the said biomarker by direct measurements, e.g., intensity values in mass spectra. Moreover, encompassed are all values or parameters which are obtained by indirect measurements specified elsewhere in this description. It is to be understood that values correlating to the aforementioned level of PSA glycoforms or parameters can also be obtained by all standard mathematical operations.

In a particular embodiment, the level of a PSA glycoform may correspond to a normalized amount of the PSA glycoform (e.g. N₆₉ glycopeptide) relative to the sum of amounts of all measured PSA glycoforms (e.g. all glycoforms as shown in Table 1A).

In a particular embodiment, the level of a PSA glycoform corresponds to a normalized signal (e.g. area under the curve) correlating with the amount of the PSA glycoform(e.g. Nee glycopeptide) which is normalized to the signal of all measured PSA glycoforms (e.g. all glycoforms as shown in Table 1A).

The level of a PSA glycoform may be used directly to compare to a reference value or may be combined with the level of one or more other PSA glycoform(s), for example as an aggressiveness score being based on a ratio or percentage of the level of one or more other PSA glycoform(s), e.g. where the level of the one or more other PSA glycoform(s) also holds some biochemical significance to the clinical condition of interest. For example, the level of a mono-antennary glycoform can be combined with that of a di-antennary glycan species to yield a ratio level. Alternatively, the levels of two or more PSA glycoform(s) can be imported into an equation to yield a score that is or can be used to generate a PCa aggressiveness score (herein aggressiveness score).

The term “assessing” as used herein refers to assessing or working out whether a subject has a particular condition or not, e.g. aggressive PCa or not. Accordingly, assessing as used herein, includes work towards determining or identifying whether or not the subject is likely to have aggressive PCa and/or whether or not the subject is likely to have an indolent prostatic disease.

As will be understood by those skilled in the art, the assessment made in accordance with the present invention, although preferred to be, may usually not be correct for 100% of the investigated subjects. The term, typically, requires that a statistically significant portion of subjects can be correctly assessed. Whether a portion is statistically significant can be determined without further ado by the person skilled in the art using various well known statistic evaluation tools, e.g., determination of confidence intervals, p-value determination, Student's t-test, Mann-Whitney test, etc. Details may be found in Dowdy and Wearden, Statistics for Research, John Wiley & Sons, New York 1983. Typically envisaged confidence intervals are at least 50%, at least 60%, at least 70%, at least 80%, at least 90%, at least 95%. The p-values are, typically, 0.2, 0.1, 0.05.

The term aggressiveness score as used herein refers to a score, e.g. numerical score, that correlates with the degree of PCa aggressiveness in the sample. It is typically determined from any mathematical combination of the determined levels optionally including other biomarkers and/or clinical information. Suitably, this score is capable to distinguish aggressive PCa from indolent prostatic disease etc. by comparing it to a reference value for said aggressiveness score indicative of a certain degree of aggressiveness. The reference aggressiveness score could be a threshold value such that any test score that falls one side or other of that value (depending on how the scoring is calibrated, e.g. whether high or low score means more aggressive) identifies or can aid in identifying the subject as likely to have an aggressive form of PCa or not. The aggressiveness score is determined by taking into consideration the combined level of the determined PSA glycoforms. As used herein, “by taking into consideration the combined level of the determined PSA glycoforms”, means that the level of each determined PSA glycoform (from the test subject, the control sample or historical control samples, as the case may be) is used in an equation involving the level of each determined PSA glycoform to yield a score which can be the aggressiveness score. By way of example, this could be the aggregate/sum of the individual levels (so, for example, aggressiveness score=level of PSA glycoform 1+level PSA glycoform 2, so that a level of x for PSA glycoform 1 and y for PSA glycoform 2 in the subject's biological “test” sample gives a combined level of x+y); Alternatively, this could be a ratio of for example PSA glycoform 1/PSA glycoform 2 (=x/y in the above example); or a ratio of for example PSA glycoform 2/PSA glycoform 1(=y/x in the above example). Alternatively, this could be some other equation involving the level of each determined PSA glycoform (e.g. if equation is: aggressiveness score=2.5 times PSA glycoform 1+1.5 times PSA glycoform 2, then using the x and y values as above would give an aggressiveness score of 2.5x+1.5y for the test subject). The appropriate equation for use in determining the aggressiveness score using any particular combination of PSA glycoforms can be determined using standard statistical analyses on appropriate reference samples.

For use in the methods of the invention the level of the one or more PSA glycoform(s) or the aggressiveness score in the subject sample (test sample) will be compared to a respective reference value capable of distinguishing subjects with aggressive PCa from those that do not have aggressive PCa. The reference value may be based on the level of the same PSA glycoform or calculation method of an aggressiveness score as the determined level or aggressiveness score. Such reference value can be referred to as the reference value for the aggressiveness score and as described herein such reference value for the aggressiveness score can be determined by comparing the level of the measured PSA glycoform seen in individuals that have aggressive forms of PCa and/or those in individuals that do not.

For example, a level of a measured PSA glycoform in a subject's test sample, on its own or when combined with the level of one or more other PSA glycoform(s) in an appropriate equation to give an aggressiveness score, can be compared to values known to be associated with aggressive PCa and/or known to be associated with individuals who do not have aggressive PCa. Usually the level in the test sample is directly or indirectly correlated with a PCa aggressiveness status and the marker level is e.g. used to determine whether or not an individual likely has aggressive PCa.

In the context of the invention the aggressiveness score may be calculated in that the individual parameters (including or consisting of the determined or received levels of PSA glycoforms) are mathematically combined. The levels may be used as such or may be mathematically transformed (e.g. by log transformation such as log 2 or log 10 transformation) for determining the score. The score may take into account one or more other factors than levels of PSA glycoform(s) including but not limited to the presence or level of one or more other biomarkers in the sample and/or one or more clinical parameters of the subject (e.g. tumour histology, smoking status, stage of disease, and/or age).

In certain embodiments of the invention, the aggressiveness score may be obtained by a weighted calculation of the level of the biomarker molecule(s) (e.g. PSA glycoform(s)) in the samples. This means that the markers may be given different weighting than others. For example, when the score takes into account the level of a PSA glycoform (e.g. mono-antennary compound 2) and the level of another PSA glycoform (e.g. di-antennary compound 9 in the sample, the score may be calculated by the following equation: Score=a*[compound 2]+b*[compound 9],wherein a and b represent the weighting factors.

Preferably, the weighting factors or coefficients (in the above example a and b have been obtained by analysing a reference population (e.g. any reference population as defined in the context of the reference value below). In embodiments, the weighting factors or coefficients may be obtained by a machine learning approach applied on a training data set obtained from samples of a reference population as defined herein in the context of the reference value and/or control sample.

A person skilled in the art is aware that the aggressiveness score and a corresponding reference value for the score can be optimized based on a reference population (e.g. any as defined in the context of the determination of a cut-off below).

In embodiments of the invention the aggressiveness score may comprise or consist of a ratio of the level of the at least one di-antennary PSA glycoform from the subject sample over the at least one mono-antennary PSA glycoform or a vice versa.

In embodiments in which two or more mono-antennary or di-antennary PSA glycoform(s) are determined, a sum of the levels of all respective mono-antennary or di-antennary can be generated and used as value for the ratio. The ratio being the sum of the levels of one category of PSA glycoform over the sum of the other category of PSA glycoform, wherein, for example PSA glycoform categories include mono-antennary forms and di-antennary forms. For example, the ratio could consist of all measured di-antennary species over all measured mono-antennary species, e.g. (A+B)/C (wherein A and B are levels of different di-antennary species and C is the level of a single monoantennary species; or (A+B)/(C+D) wherein D is a second monoantennary species). Similarly if the ratio is all measured mono-antennary species over all measured di-antennary species, it could be C/(A+B) or (C+D)/(A+B). Such ratio can then be used to determine whether or not the PCa is aggressive PCa.

Thus, in embodiments in which two or more mono-antennary or di-antennary PSA glycoforms are determined, a sum of the respective monoantennary or di-antennary levels would be calculated and used as the value for the ratio.

In one embodiment, the aggressiveness score may be a binary score and the corresponding reference value may also be binary. “Binary” means that the score contains two values, e.g. a first value being the level of the one or more di-antennary PSA glycoforms determined or received or a value derived therefrom and a second value being the level of the one or more mono-antennary PSA glycoforms or a value derived therefrom. A “value derived therefrom” can, for example, be a value obtained by a mathematical operation. The “value derived therefrom” is preferably directly proportional to the respective level. The values of the binary reference value may be obtained as described below for individual PSA glycoforms.

Comparing a binary score to a binary cut-off score means comparing the first value of the determined binary score to the first value of the reference value of the binary score and the second value of the determined binary score to the second value of reference value of the binary score. In embodiments, the aggressiveness score is a binary score comprising a first value corresponding to the level of the at least one mono-antennary PSA glycoform and a second value corresponding to the level of the at least one di-antennary PSA glycoform and the reference value for the aggressiveness score comprises a reference value for the first value and a reference value for the second value. In these embodiments, if the level of the at least one mono-antennary PSA glycoform (i.e. the first value of the binary score) is decreased compared to reference value for the first value and the level of the at least one di-antennary PSA glycoform (i.e. the second value of the binary score) is increased compared to reference value for the second value, the subject is determined to have an aggressive form of PCa. Vice versa, if only one or none of the first and second value of such binary scores fulfils the aggressiveness criteria, the subject is determined to have non-aggressive prostatic disease.

The term “comparing” as used herein has its usual meaning, e.g. the act of examining things to see if they are similar or different. In the context of the present invention that includes comparing the level of a PSA glycoform in the sample from the subject with the reference level of the PSA glycoform, or e.g. comparing the calculated aggressiveness score (e.g. calculated based on the mathematical combination of the levels of various PSA glycoforms measured using, for example, weighted calculation, calculating aggregate (e.g. summed) levels or building a ratio), to the reference value for such aggressiveness score. It is to be understood that comparing as used herein usually refers to a comparison of corresponding parameters, values or scores, e.g., an absolute amount is compared to an absolute reference amount while a concentration is compared to a reference concentration or an intensity signal obtained from the biomarker in a sample is compared to the same type of intensity signal obtained from a reference sample or a collated value (e.g. aggressiveness score) from inputting the value of the levels of multiple biomarkers (e.g. PSA glycoforms) into an equation is compared to the reference value for the collated the levels from the same biomarkers (e.g. obtained from a reference control sample or a reference cohort). As described herein the reference value can also be obtained from multiple control or reference samples, e.g. from a reference cohort with known prostate (cancer) disease status (e.g. Gleason score). The comparison may be carried out manually or computer-assisted. Thus, the comparison may be carried out by a computing device. The value of the measured or detected level of the biomarker in the sample from the subject and the reference level can be, e.g., compared to each other and the said comparison can be automatically carried out by a computer program executing an algorithm for the comparison. The computer program carrying out the said evaluation will provide the desired assessment in a suitable output format. For a computer-assisted comparison, the value of the measured level may be compared to values corresponding to suitable references which are stored in a database by a computer program. The computer program may further evaluate the result of the comparison, i.e. automatically provide the desired assessment in a suitable output format. For a computer-assisted comparison, the value of the measured level may be compared to values corresponding to suitable references which are stored in a database by a computer program. The computer program may further evaluate the result of the comparison, i.e. automatically provides the desired assessment in a suitable output format.

The term “reference value” as used herein, refers to a known (e.g. predetermined) value against which a test value can be compared and so is a value to distinguish between two states (e.g. yes, no; disease/healthy; aggressive PCa/not aggressive PCa). There are essentially two alternatives to determine a reference value: 1) control sample (can be an internal or external sample or a pool of samples); 2) multiple samples of a reference population with known disease status (e.g. aggressive PCa and/or indolent prostatic disease).

Suitably a reference value can be determined from one or more control samples which is or has/have been analysed and determined in a substantially identical manner as the test sample of interest and whose information is compared to that of the test sample of interest. In one embodiment, the control sample is obtained from a control subject that does not have aggressive PCa. In another embodiment, the control sample is obtained from a subject that has indolent prostatic disease, such as indolent (slow growing) PCa or BPH. In another embodiment, the control sample is obtained from a control subject that has an aggressive form of PCa.

Control samples can be evaluated substantially at the same time as the test sample and so be used as a direct contemporaneous comparator, or they can be from historical samples. The reference value can therefore be determined alongside that of the test sample (contemporaneously) or pre-determined. Pre-determined reference values are preferred. The values from the one or more control samples thereby provides a standard allowing for the evaluation of the information obtained from the sample of interest (test sample). In particular embodiments, the control sample is the same sample type as that of the sample of interest. For example, if the sample of interest is urine, the control sample may be urine. A control sample may be derived from a body fluid obtained from a healthy individual in particular urine, serum or plasma for a non-invasive test, thereby providing a standard of a healthy or defined disease status of a tissue, organ or individual. Alternatively, the control sample may be obtained from at least one individual that has aggressive PCa from the same species, such as one with cancer with a Gleason score >7. Alternatively, the control sample may be obtained from at least one individual that does not have aggressive PCa from the same species, or a sample obtained from at least one individual having a non-cancerous prostatic disorder, such as BPH or prostatitis, from the same species. In a particular embodiment, the control sample can be from an individual with benign PCa with a Gleason score <7. The individual can be the same age or in the same state or condition of health as the subject from which the test sample is obtained. Differences between the status of the control sample and the status of the test sample of interest may be indicative of the presence of the disease or risk of disease development or the presence or further progression of such disease or disorder. A control sample may be derived from an abnormal or diseased tissue, organ, body fluid or individual (e.g. one with indolent prostatic disease) thereby providing a standard of a diseased status of a tissue, organ or individual. Differences between the status of the abnormal reference sample and the status of the sample of interest may be indicative for PCa aggressiveness. A reference sample may also be derived from the same tissue, organ, body fluid or individual as the sample of interest but has been taken at an earlier time point. Differences between the status of the earlier taken reference sample and the status of the sample of interest may be indicative of the progression of the disease, i.e. a bettering or worsening of the disease overtime.

The control sample may be an internal or an external control sample. An internal control sample is used, i.e. the marker level(s) is(are) assessed in the test sample as well as in one or more other sample(s) taken from the same subject to determine if there are any changes in the level(s) of said marker(s). For an external control sample the level of a marker in a sample derived from the individual is compared to its level in an individual known to suffer from, or known to be at risk of, a given condition; or an individual known to be free of a given condition, i.e., “normal individual”.

It will be appreciated by the skilled artisan that such external control sample may be obtained from a single individual or may be obtained from a reference population that is age-matched. Typically, samples from 100 well-characterized individuals from the appropriate reference population are used to establish a “reference value”. However, reference population may also be chosen to consist of, for example, 20, 30, 50, 200, 500 or 1000 individuals.

Thus, alternatively, a reference value can be determined based on samples obtained from a reference population, e.g. samples of a reference population with known prostate disease and PCa aggressiveness status. A skilled person can establish a reference value for the level of a PSA glycoform or a reference value for an aggressiveness score based on a representative reference population. Using a reference population the reference value can be determined such that the level or score value set as reference value divides the reference population into aggressive PCa and indolent prostate disease with a certain sensitivity and specificity. The reference value may be selected differently depending on whether higher sensitivity at cost of lower specificity is desired or vice versa. The level or score separating, e.g. aggressive PCa from non-aggressive PCa can be determined using commonly known statistic methods such that a desired sensitivity and specificity is defined. The reference value that is used in the comparison with the test sample may be a value that is calculated as an average or median of more than one (e.g. two or more, five or more, ten or more, a group etc.) of control samples. Alternatively, the control sample may be a sample that originated from (i.e. is a mix of) more than one (e.g. two or more, five or more, ten or more, a group etc.) individual that is not suffering from PCa (or that has BPH).

The reference value is typically determined from statistical assessment of biomarker readouts/levels from multiple (e.g. >49, >99, >249, >499, >999) subject samples (e.g. from a reference population) with or without the particular state to be determined (e.g. aggressive PCa). The reference value can be a value for the level of a single biomarker (i.e. an individual PSA glycoform) or an aggressiveness score value derived from the individual levels of a combination of biomarkers (i.e. two or more PSA glycoforms, as described herein), which levels may have been applied to an algorithm or equation to give a reference value that can be used when measuring and relying on said particular combination of biomarkers (e.g. combination of PSA glycoforms). Typically, the reference value achieves a particular statistical threshold of significance, e.g. of quantiles in the case or control distributions. For example, to determine the reference value from a certain control cohort (BPH or indolent PCa), a certain percentile may be determined (e.g. 90%, 95%, 95.7 or 97%) from the measurement values of this control cohort. E.g. when the 90^{th} percentile is used, then 90% of the control samples have values below this cut-off, which reduces the number of false positive predictions.

In one example, a reference value is the average or median level of the one or more PSA glycoform(s) taken from a group or population of individuals suffering from aggressive PCa from the same species. In one example, the reference value is the average or median, taken from a group or population of individuals that are not suffering from PCa. For example, the reference value may be calculated as the average or median, taken from a group or population of individuals that have aggressive PCa, and/or indolent prostatic disease (such as indolent PCa, and/or BPH, and/or prostatitis). The individual or the population of individuals can be the same age or in the same state or condition of health as the subject from which the test sample is obtained.

In a specific embodiment, the reference population for establishing a reference value (e.g. for a level of a PSA glycoform or an aggressiveness score) is derived from healthy individuals. In another embodiment, the reference population may contain healthy individuals and those with non-aggressive and aggressive forms of PCa. In another embodiment, the reference population may comprise individuals with non-aggressive (BPH and/or indolent) and aggressive PCa. In another embodiment, the reference population may comprise individuals with indolent prostatic disease. In another embodiment, the reference population may be as the one used in the examples. Preferably, the reference value in each of these embodiments is a pre-determined reference value established from historical control samples, e.g. derived from a reference population.

The reference value may also be a reference value for an aggressiveness score (if an aggressiveness score is determined) generated from measurements of the level of two or more PSA glycoforms; again from a group or population of individuals with known PCa status. Such levels may be input into an equation to generate a score (e.g. aggressiveness score). The equation could be a ratio or aggregate (e.g. sum) or any other equation where the level of each PSA glycoform measured is used in the equation and wherein the output provides a score/value which distinguishes whether the individual falls into a particular category or not (e.g. likely has aggressive PCa, likely does not have aggressive PCa).

As used herein, an individual or subject that “does not have PCa” is one that has histologically normal-appearing prostate tissue. Methods for histologically testing prostate tissue and identifying whether an individual has histologically normal-appearing prostate tissue are well known in the art, see for example Litwin M S and Tan H J., The Diagnosis and Treatment of Prostate Cancer: A Review. JAMA. 2017 Jun. 27; 317(24):2532-254. A control sample that is obtained from an individual that does not have PCa in this context therefore refers to a biological fluid sample (e.g. a blood or urine sample, as appropriate) that has been obtained from an individual of the same species, where the individual has histologically normal-appearing prostate tissue. Examples of individuals that do not have PCa include individuals with BPH, prostatitis and/or an enlarged prostate.

Benign prostatic hyperplasia (BPH; also known as benign prostate enlargement) is a medical condition that is common in men aged over 50. It is a condition that can affect how urine is passed but is not a cancer and does not result in an increased risk of developing PCa.

Accordingly, as used herein, an individual/subject/patient that has “benign prostatic hyperplasia” or “BPH” is an one that has an enlarged prostate with histologically normal-appearing prostate tissue. Methods for histologically testing prostate tissue and identifying whether an individual has BPH are well known in the art, see for example Chughtai et al. Benign prostatic hyperplasia. Nat Rev Dis Primers. 2016 May 5; 2:16031. A control sample that is obtained from an individual that has BPH in this context therefore refers to a biological fluid sample (e.g. a blood or urine sample, as appropriate) that has been obtained from an individual of the same species, where the individual has an enlarged prostate with histologically normal-appearing prostate tissue.

Prostatitis is the name given to a set of symptoms which are thought to be caused by an infection or by inflammation of the prostate gland. Prostatitis is not a form of PCa. It is a common condition which can affect men of any age, but it is most common in younger and middle aged men, typically between 30 and 50. Prostatitis can cause a wide range of symptoms, which vary from man to man. Common symptoms include problems passing urine and pain or discomfort around the testicles, back passage or lower abdomen. There are four types of prostatitis, Chronic pelvic pain syndrome (CPPS), Acute bacterial prostatitis, Chronic bacterial prostatitis and Asymptomatic prostatitis.

A control sample that is obtained from an “individual with prostatitis” therefore refers to a biological fluid sample (e.g. a blood or urine sample, as appropriate) that has been obtained from an individual of the same species, where the individual has been diagnosed with one of the above forms of prostatitis.

An enlarged prostate is an increase in the size of the prostate that is not caused by cancer. The medical term for an enlarged prostate is benign prostatic enlargement (BPE). It is also referred to as benign prostatic hyperplasia (BPH), which is described in more detail above.

The term “measurement” or “measuring” or “determining” in the context of analysis preferably comprises a qualitative, a semi-quantitative or a quantitative measurement. The term “determining” is also used in the context of making a decision as to the status of the subject, e.g. identify whether they have aggressive PCa or not. In this context the term “identifying” may be used.

As will be understood by those skilled in the art, the determinations made in accordance with the present invention, although preferred to be, may usually not be correct for 100% of the investigated subjects. The term, typically, requires that a statistically significant portion of subjects can be correctly assessed. Whether a portion is statistically significant can be determined without further ado by the person skilled in the art using various well known statistic evaluation tools, e.g., determination of confidence intervals, p-value determination, Student's t-test, Mann-Whitney test, etc.. Details may be found in Dowdy and Wearden, Statistics for Research, John Wiley & Sons, New York 1983. Typically envisaged confidence intervals are at least 50%, at least 60%, at least 70%, at least 80%, at least 90%, at least 95%. The p-values are, typically, 0.2, 0.1, 0.05. As such, as used herein when determining whether a subject has aggressive PCa, the determining/determination may be for a likelihood of someone having aggressive PCa.

The terms “cancer” and “cancerous” refer to or describe the physiological condition that is typically characterized by unregulated cell growth. Examples of cancer include cancer of the urogenital tract, such as PCa. As used herein, the term “prostate cancer (PCa)” refers to all stages and all forms of cancer arising from the tissue of the prostate gland.

As used herein the term “prostatic disease” refers to any and all diseases or disorders of the prostate, such as PCa (whether that is slow growing/indolent or aggressive), prostatitis (including bacterial prostatitis—acute or chronic bacterial infection and non-bacterial prostatitis), inflamed prostate, also known as chronic pelvic pain syndrome (CPPS) and BPH.

As used herein, “grey zone”, “PSA grey zone”, “grey area”, or “PSA grey area” as used herein, refers to the range 2-10 ng/ml total PSA in the subject's serum. As described elsewhere herein, PSA testing is a non-definitive measure and subjects with total serum PSA levels in the range 2-10 ng/ml may or may not have PCa. The methods of the present invention are particularly suitable for subjects whose serum PSA levels are in this “grey zone”. Suitably, the methods of the invention are conducted on a sample from a subject whose serum PSA levels are in this “grey zone”.

Methods of diagnosing and staging PCa are well known in the art. For example, according to the tumour, node, metastasis (TNM) staging system of the American Joint Committee on Cancer (AJCC), AJCC Cancer Staging Manual (7th Ed., 2010), the various stages of PCa are defined as follows: Tumour: T1: clinically inapparent tumour not palpable or visible by imaging, T1a: tumour incidental histological finding in 5% or less of tissue resected, T1b: tumour incidental histological finding in more than 5% of tissue resected, T1c: tumour identified by needle biopsy; T2: tumour confined within prostate, T2a: tumour involves one half of one lobe or less, T2b: tumour involves more than half of one lobe, but not both lobes, T2c: tumour involves both lobes; T3: tumour extends through the prostatic capsule, T3a: extracapsular extension (unilateral or bilateral), T3b: tumour invades seminal vesicle(s); T4: tumour is fixed or invades adjacent structures other than seminal vesicles (bladder neck, external sphincter, rectum, levator muscles, or pelvicwall). Node: N0: no regional lymph node metastasis; N1: metastasis in regional lymph nodes. Metastasis: M0: no distant metastasis; M1: distant metastasis present.

The Gleason Grading system is also commonly used to help evaluate the prognosis of men with PCa. Together with other parameters, it is incorporated into a strategy of PCa staging, which predicts prognosis and helps guide therapy. A Gleason “score” or “grade” is given to PCa based upon its microscopic appearance. Tumours with a low Gleason score (e.g. Gleason score of ≤6) typically grow slowly enough that they may not pose a significant threat to the patients in their lifetimes. These patients are monitored (“watchful waiting” or “active surveillance”) over time. Cancers with a higher Gleason score are more aggressive and have a worse prognosis, and these patients are generally treated with surgery (e.g., radical prostatectomy) and, in some cases, therapy (e.g., radiation, hormone, ultrasound, chemotherapy, immunotherapy). Gleason scores (or sums) comprise grades of the two most common tumour patterns. These patterns are referred to as Gleason patterns 1-5, with pattern 1 being the most well-differentiated. Most have a mixture of patterns. To obtain a Gleason score or grade, the dominant pattern is added to the second most prevalent pattern to obtain a number between 2 and 10. The Gleason Grades include: G1: well differentiated (slight anaplasia) (Gleason 2-4); G2: moderately differentiated (moderate anaplasia) (Gleason 5-6); G3-4: poorly differentiated/undifferentiated (marked anaplasia) (Gleason 7-10).

As used herein, “indolent prostatic disease” refers to subjects/patients that have a Gleason score of ≤6.

As used herein “aggressive prostate cancer” or “aggressive PCa” refers to subjects/patients refers to patients that have Gleason score of >7. Such a score could, for example be 3+4.

The methods described herein may be used on samples from patients with a prostatic disease or that have an increased risk of developing PCa. In this context, the phrase “increased risk” indicates that the subject has a higher level of risk (or likelihood) that they will experience a particular clinical outcome. A subject may be at increased risk of developing PCa if there is a family history of the disease. A subject may be may at increased risk of developing PCa if they possess genetic mutations which predispose to development of PCa, e.g. BRCA1 and BRCA2 and HPC1, androgen receptor mutations and TMPRSS2:ERG gene fusion.

A biomarker is a characteristic that is objectively measured and evaluated as an indicator of a biological state of said system (e.g. normal biological processes, pathogenic processes or pharmacological responses to a therapeutic intervention). A biomarker can be any kind of molecule present in a living organism, such as a nucleic acid (DNA, mRNA, miRNA, rRNA etc.), a protein, polypeptide, peptide, immunologically detectable fragment thereof (cell surface receptor, cytosolic protein etc.), a metabolite or hormone (blood sugar, insulin, estrogen, etc.), a molecule characteristic of a certain modification of another molecule (e.g. sugar moieties or phosphoryl residues on proteins, methyl-residues on genomic DNA). Suitably, the biomarker is differentially present in a sample taken from a subject having a disease or disease state as compared with a subject not having the disease or disease state. A biomarker is differentially present if the mean or median level of the biomarker in the different groups is calculated to be statistically significant. Common tests for statistical significance include, among others, t-test (e.g., student t-test), ANOVA, Kruskal-Wallis, Wilcoxon, Mann-Whitney, Receiver Operating Characteristic (ROC curve), accuracy and odds ratio. Biomarkers, alone or in combination, provide measures of relative risk that a subject belongs to one phenotypic status or another. Therefore, they are useful as markers for disease (diagnostics), therapeutic effectiveness of a drug and drug toxicity.

Typically, the biomarkers referred to herein are N-linked glycans that are attached to PSA protein via a nitrogen atom to the asparagine. A glycan is a carbohydrate- or sugar-based unit composed of monosaccharides linked by glycosidic bonds. The PSA glycoforms herein are biomarkers.

The term “disease” and “disorder” are used interchangeably herein, and refer to an abnormal condition, especially an abnormal medical condition such as an illness or injury, wherein a tissue, an organ or an individual/subject is not able to efficiently fulfil its function anymore. Typically, but not necessarily, a disease is associated with specific symptoms or signs indicating the presence of such disease. The presence of such symptoms or signs may thus, be indicative for a tissue, an organ or an individual suffering from a disease. An alteration of these symptoms or signs may be indicative for the progression of such a disease. A progression of a disease is typically characterised by an increase or decrease of such symptoms or signs which may indicate a “worsening” or “bettering” of the disease. The “worsening” of a disease is characterised by a decreasing ability of a tissue, organ or organism to fulfil its function efficiently, whereas the “bettering” of a disease is typically characterised by an increase in the ability of a tissue, an organ or an individual to fulfil its function efficiently. A tissue, an organ or an individual being at “risk of developing” a disease is in a healthy state but shows potential of a disease emerging. Typically, the risk of developing a disease is associated with early or weak signs or symptoms of such disease. In such case, the onset of the disease may still be prevented by treatment. Examples of a disease include but are not PCa and prostatitis.

A “glycan” or “carbohydrate” refers to a polymer that consists of monosaccharide residues or sugar moieties that are found attached to proteins as in glycoproteins, proteoglycans and lipids. Glycans can be linear or branched. Glycans can be found covalently linked to non-saccharide moieties, such as lipids or proteins. Binding to proteins typically occurs via a nitrogen (N-linkage)- or oxygen atom (O-linkage). The covalent conjugates comprising glycans are termed e.g. glycosylated polypeptides, glycoproteins, glycopeptides, peptidoglycans, proteoglycans, glycolipids, and lipopolysaccharides. Glycans exist also in free form (i.e., separate from and not associated with another moiety).

As used herein, the term “PSA glycoform”, “PSA glycoform species”, “PSA glycopeptide”, “PSA glycan” or are used interchangeably and refers, when in the singular, to one particular type of glycan structure that is attached to PSA protein or a peptide thereof. When in the context of the plural it refers to a multitude of PSA glycoforms/glycoform species. The term “glycosylation marker” or “glycan biomarker” may also be used to denote the particular glycan species bound to PSA whose level is altered, either enhanced or decreased, in certain disease states (e.g. indolent prostatic disease or aggressive PCa) and so can be used, including measured, in the methods of the invention.

Prostate specific antigen (PSA) has a single site on which N-linked glycosylation occurs, namely at amino acid residue 69 (numbering according to Kabat, E. A., et al., Sequences of Proteins of Immunological Interest, 1991). A protease can be used to cleave the PSA protein into smaller peptides, ideally wherein just one unique peptide has the asparagine at position 69 (so all the different glycoforms are on the same length peptide), herein referred to as N₆₉ comprising glycopeptide or N₆₉ glycopeptide. The cleavage of PSA with trypsin will release the N₆₉K dipeptide. The cleavage of PSA with Arg-C (clostripain) will release N₆₉KSVILLGR peptide (SEQ ID NO: 3). Thus, any glycan attached at N₆₉ is presented as a glycopeptide (N₆₉ glycopeptide, e.g. N₆₉K glycopeptide or N₆₉KSVILLGR glycopeptide) and so the level of each PSA-glycan moiety detected according to the methods of the invention can be determined by relative quantification of the level of each N₆₉K-glycoform (PSA-glycan species or PSA-glycan moiety).

As used herein, the term “N₆₉ glycopeptide” or “N₆₉ comprising glycopeptide” relates to any peptide (e.g. 20 amino acids or shorter) derived from PSA (e.g. by protease digestion) comprising the residue corresponding to N₆₉ of PSA and having a N-glycan (e.g. a specific gylcoform) attached to N₆₉.

The glycan/carbohydrate/glycoform species or moieties of the present invention will be described with reference to commonly used nomenclature for the description of oligosaccharides (e.g. see Glycobiology 25: 1323-1324, 2015 and Glycobiology 29:620-624, 2019; and legend beneath Table 1A herein) A review of carbohydrate chemistry which uses this nomenclature is found in Hubbard, S. C, and Ivatt, R. J., Ann. Rev. Biochem. 50: 555-583, 1981. Examples of PSA glycoforms that can be used in the present invention are identified in Table 1.

The term “glycosylation” means the attachment of sugar moieties (e.g. polysaccharides) to a protein. Glycosylation is a form of co-translational and post-translational modifications. Suitably, the polysaccharide consists of from two or moresimple sugars linked together via glycosidic bonds. Most glycosylation in mammalian cells are N- or O-linked to the protein. Typically, the polysaccharide is either bound via the OH group of a serine or a threonine (0-linked to produce an O-glycosylated polypeptide) or via the amide group (NH2) of asparagine (N-linked to produce an N-glycosylated polypeptide). The glycosylation of PSA is N-linked.

The terms “glycosylated polypeptide”, “glycoprotein”, “glycosylated peptide”, and “glycopeptide”, which are used interchangeably within this application refer to proteins or polypeptides or peptides having at least two amino acids wherein at least one amino acid has a covalently attached glycan or polysaccharide. In the context of a glycosylated peptide of PSA, in one embodiment it is a N₆₉K glycopeptide.

The term “glycoform” denotes a protein, polypeptide or peptide with a specific type and distribution of polysaccharides attached to, i.e. two identical peptides/polypeptides would be of the same glycoform if they comprise glycans with the same number, kind, and sequence of monosaccharides; whereas two identical peptides (identical in terms of amino acid sequence) would be different glycoforms if they comprise glycans with a different number, kind, and sequence of monosaccharides. In the context of a glycoform of PSA this is suitably a N₆₉K glycopeptide with a particular glycan moiety attached. Glycoforms are also glycosylated polypeptides.

As used herein, the term “mass spectrometry” or “MS” refers to an analytical technique to identify compounds by their mass. Typically, a sample is analysed by generating gas phase ions from the sample, which are then separated according to their mass-to-charge ratio (m/z) and detected. MS technology generally includes (1) ionizing the compounds to form charged compounds; (2) calculating a mass-to-charge ratio (m/z). and (3) determining the molecular weight of the charged compounds. The compounds may be ionized and detected by any suitable means. A “mass spectrometer” generally includes an ionizer and an ion detector. In general, one or more molecules of interest are ionized, and the ions are subsequently introduced into a mass spectrometric instrument where, due to a combination of magnetic and electric fields, the ions follow a path in space that is dependent upon mass (“m”) and charge (“z”).

Methods of generating gas phase ions from a sample include electrospray ionization (ESI), matrix-assisted laser desorption-ionization (MALDI), surface-enhanced laser desorption-ionization (SELDI) and chemical ionization. Separation of ions according to their m/z ratio can be accomplished with any type of mass analyser, including quadrupole mass analysers (Q), time-of-flight (TOF) mass analysers, magnetic sector mass analysers, 3D and linear ion traps (IT), Fourier-transform ion cyclotron resonance (FT-ICR) analysers, and combinations thereof (for example, a quadrupole-time-of-flight analyser, or Q-TOF analyser). Prior to ionisation, the sample may be subjected to one or more dimensions of chromatographic or electrophoretic separation, for example, one or more dimensions of liquid or size exclusion chromatography.

As used herein, the term “matrix-assisted laser desorption ionization” or “MALDI” refers to an ionization technique that uses a laser energy absorbing matrix to create ions from large molecules with minimal fragmentation. MALDI methodology is a three-step process. First, the sample is mixed with a suitable matrix material and applied to a metal plate. Second, a pulsed laser irradiates the sample and thirdly, the analyte molecules are ionized by being protonated (H⁺ addition) or deprotonated (H⁺ removal) in the hot plume of ablated gases. They can then be accelerated into whichever mass spectrometer is used to analyse them

As used herein, the term “electrospray ionization” or “ESI,” refers to methods in which a high voltage is applied to a liquid to create an aerosol of very small droplets of solution in solvent vapor. This mist of droplets flows through an evaporation chamber. Based on current understanding, as the droplets get smaller the electrical surface charge density increases until such time that the natural repulsion between like charges causes ions as well as neutral molecules to be released. ESI is different from other ionization methods in that it may produce multiple-charged ions, effectively extending the mass range of the analyser. Heated ESI is similar but includes a heat source for heating the sample while in the capillary tube. Mass spectrometry using ESI is called electrospray ionization mass spectrometry (ESI-MS).

Prostate-specific antigen (PSA) (also known as kallikrein related peptidase 3, APS, prostate-specific antigen (PSA), hK3, KLK2A1, P-30 antigen, gamma-seminoprotein, kallikrein-3, semenogelase, seminin NP_001025218.1 (EC 3.4.21.77), NP_001025219.1 (EC 3.4.21.77), NP_001639.1 (EC 3.4.21.77) is a glycoprotein enzyme encoded by the KLK3 gene. KLK3 is a member of the kallikrein-related peptidase family and is secreted by the epithelial cells of the prostate gland. PSA mRNA is translated as an inactive 261 amino acid preproPSA precursor. PreproPSA has 24 additional residues that constitute the pre-region (the signal polypeptide) and the propolypeptide. Release of the propolypeptide results in the 237—amino acid, mature extracellular form, which is enzymatically active. PSA is organ-specific and, as a result, it is produced by the epithelial cells of BPH tissue, primary PCa tissue, and metastatic PCa tissue.

PSA is concentrated in prostatic tissue, and serum PSA levels are normally very low. Disruption of the normal prostate architecture, for example by prostatic disease, inflammation or trauma, releases greater amounts of PSA into the circulation system (e. blood). PSA measurement is used to detect potential problems in the prostate gland and to follow the progress of PCa therapy. However, PSA is not a specific marker for PCa, since its levels increase due to other conditions, including BPH and prostatitis, and PSA levels are also known to be affected by factors such as trauma, medication, urologic manipulation and inflammation. PSA is present in small quantities in serum of men with a healthy prostate, but is often elevated in subjects with PCa and other prostate disorders. It has been demonstrated that between 40 and 45% of the variability in PSA levels in the general population is due to inherited factors (Gudmundsson et al. Sci Transl Med. 2010 December 15; 2(62): 62ra92. doi:10.1126/scitranslmed.3001513).

As used herein, the term “sample” or “biofluid sample”, “test sample”, and variations thereof, encompass biological samples obtained from a patient or subject, which may comprise blood, plasma, serum, urine, prostatic fluid (such as a prostatic secretion sample), semen, saliva or sputum. For the purposes described herein, the sample is, or comprises, a biological fluid (also referred to herein as a bodily fluid) sample. Suitably, the sample is an urine sample e.g., digital rectal examination (DRE) urine sample. Suitably, the sample is or has been obtained or derived from a subject. A DRE urine sample is one that is collected immediately after DRE. By way of example, this may involve conducting a DRE examination wherein some pressure is applied to the left and right lobes of the prostate before urination and the first 50 mL (approximately) of the initial urine stream of urine is collected. This urine is referred to as DRE urine.

As used herein, “provide”, “obtain” or “obtaining” in the context of a sample, can be any means whereby one comes into possession of the sample by “direct” or “indirect” means. Directly obtaining a sample means performing a process (e.g., performing a physical method such as extraction) to obtain the sample. Indirectly obtaining a sample refers to receiving the sample from another party or source (e.g., a third party laboratory that directly acquired the sample). In certain embodiments, the methods of the invention are carried out on samples that have been obtained indirectly. In certain embodiments, the methods of the invention include an additional step of directly obtaining the sample from the subject.

The methods provided herein may comprise providing a biological fluid sample (for example a blood sample or a urine sample) from a subject.

In general, the methods described herein are ex vivo methods that are performed using a sample that has already been obtained from the subject (i.e. the sample is provided for the method, and the steps taken to obtain the sample from the subject are not included as part of the method).

For the avoidance of doubt, the term “ex vivo” has its usual meaning in the art, referring to methods that are carried out in or on a sample obtained from a subject in an artificial environment outside the body of the subject from whom the sample has been obtained. In particular embodiments, the subjects biofluid sample has been previously isolated/taken from the subject and the sample isolation does not form part of the method of the invention. In another embodiment, the sample isolation can be a step of the method of the invention.

The term “subject” as used herein refers to an animal, more preferably a mammal, for example, humans, chimpanzees, Rhesus monkeys, dogs, cows, horses, cats, rats, or mice, provided that they also have a prostate. The subject is preferably a mammal, and most preferably a human. Suitably, the subject is male, such as a male human.

The subject may be referred to herein as a patient. The terms “subject”, “individual”, and “patient” are used herein interchangeably. The subject can be symptomatic (e.g., the subject presents symptoms associated with PCa or prostatic disease), or the subject can be asymptomatic (e.g., the subject does not present symptoms associated with PCa or prostatic disease).

“Watchful waiting” or “active surveillance”, as used herein refers to the strategy of managing a patient with prostatic disease. PCa is often slow growing and may never cause any problems or symptoms. Many treatments and diagnostic procedures for PCa can cause side effects. For some men these side effects may be long-term and may have a big impact on their lives. Therefore, watchful waiting is a way of monitoring PCa that is not causing any symptoms or problems over time, and avoiding treatment unless they get symptoms. Many men on watchful waiting will never need any treatment for their PCa.

According to a first aspect of the invention there is provided a method comprising:
(a) determining the level of at least one mono-antennary PSA glycoform and the level of at least one di-antennary PSA glycoform in a biofluid sample from the subject;
(b) calculating an aggressiveness score for the subject based on the levels determined in (a);
(c) comparing the subject's aggressiveness score in (b) to a reference value for said aggressiveness score; and
(d) using the comparison in (c) to aid in determining whether the subject has an aggressive form of prostate cancer or not.

The method can be used for different purposes. For example, to determine or aid in determining whether or not a subject has an aggressive form of PCa; or to determine or aid in determining whether or not a subject has indolent prostatic disease.

Suitably, there is provided a method to aid in determining whether or not a subject has an aggressive form of prostate cancer, comprising:
(a) determining the level of at least one mono-antennary PSA glycoform and the level of at least one di-antennary PSA glycoform in a biofluid sample from the subject;
(b) calculating an aggressiveness score for the subject based on the levels determined in (a);
(c) comparing the subject's aggressiveness score in (b) to a reference value for said aggressiveness score; and
(d) using the comparison in (c) to aid in determining whether the subject has an aggressive form of prostate cancer or not.

Suitably, the comparison and/or calculated score in (b) determines whether the subject has an aggressive form of prostate cancer or not.

In a particular embodiment, the reference aggressiveness score is or has been determined by taking into consideration the combined level of the determined PSA glycoforms.

In a particular embodiment, the subject's aggressiveness score is determined from or takes into account the individual levels of the PSA glycoforms.

In a particular embodiment, the subject's aggressiveness score is determined by taking into consideration a combined level of two or more determined PSA glycoforms, wherein at least one is a mono-antennary PSA glycoform and at least one is a di-antennary PSA glycoform.

In a particular embodiment, the subject's aggressiveness score is determined by taking into consideration a combined level of two or more determined PSA glycoforms having a shared structural feature (e.g. two or more mono-antennary or two or more di-antennary PSA glycoforms).

In a particular embodiment, the subject's aggressiveness score may comprise or be determined by building the ratio of the level of one determined PSA glycoform against the other (e.g. ratio from a mono-antennary PSA glycoform and a di-antennary PSA glycoform or vice versa)

In a variant of this first aspect of the invention, there is provided a method to aid in determining whether or not a subject has an aggressive form of prostate cancer, comprising:
(a) determining the level of at least one mono-antennary PSA glycoform and the level of at least one di-antennary PSA glycoform in a biofluid sample from the subject;
(b) comparing the level of each PSA glycoform to control or reference values for said PSA glycoform and/or calculating a score for assessing whether the subject has aggressive PCa by taking into consideration the level of each PSA glycoform; and/or comparing the value of the combined levels of each measured PSA glycoform to control or reference values for said combined PSA glycoform and/or calculating a score for assessing whether the subject has aggressive PCa by taking into consideration the combined level of each determined PSA glycoform; and
(c) using the comparison and/or calculated score in (b) to aid in determining whether the subject has an aggressive form of prostate cancer or not.

In particular embodiments, the subject is an animal, more preferably a mammal, for example, humans, chimpanzees, Rhesus monkeys, dogs, cows, horses, cats, rats, or mice, provided that they also have a prostate. The subject is preferably a mammal, and most preferably a human. Suitably, the subject is male, such as a male human.

In a particular embodiment, if the subject is not determined to have an aggressive form of PCa they are determined to have or are likely to have indolent prostatic disease.

In particular embodiments, the at least one mono-antennary PSA glycoform and at least one di-antennary PSA glycoform are selected from the mono-antennary PSA glycoforms identified as compounds 2, 3, 4 or 5, and the di-antennary PSA glycoforms identified as compounds 7, 9, 10, 11, 13, 16, 18, 21, 24 and 25, in Table 1A and B.

In other particular embodiments, the at least one mono-antennary PSA glycoform and the at least one di-antennary PSA glycoform are selected from the mono-antennary PSA glycoforms identified as compound 2, 3 and 4 and the di-antennary PSA glycoforms identified as compound 7, 9, 18, 11, 13, 24, in Table 1A and B.

In yet other particular embodiments, the at least one mono-antennary PSA glycoform and the at least one di-antennary PSA glycoform are selected from the mono-antennary PSA glycoforms identified as 2, 3, 4 and 5 and the di-antennary PSA glycoforms identified as compound 7, 9, 24 and 18, in Table 1A and B.

In particular embodiments, the at least one mono-antennary PSA glycoform is selected from the group consisting of: H4N3S_{2,6}1 (compound 2); H4N3F1S_{2,6}1 (compound 3); H4N3F1S_{2,3}1 (compound 4); and H3N4F1S_{2,6}1 (compound 5).

In particular embodiments, the at least one di-antennary PSA glycoform is selected from the group consisting of: H4N4F1S_{2,6}1 (compound 7); H5N4F1S_{2,6}1 (compound 9); H5N4F1S_{2,3}1 (compound 10); H4N5F1S_{2,6}1 (compound 11); H5N4S_{2,3}1S_{2,6}1 (compound 13); H4N5S_{2,3}1S_{2,6}1 (compound 16); H4N5F1S_{2,3}1S_{2,6}1 (compound 18); H4N5S_{2,3}1(SO₃)1 (compound 21); H4N5F1S_{2,6}1(SO₃)1(compound 24); and H4N5F1 S_{2,6}1 (SO₃)1 (compound 25).

In particular embodiments, the at least one di-antennary PSA glycoform is a di-antennary PSA glycoform comprising alpha 2,6-linked sialylation. Such di-antennary and alpha 2,6-linked sialylated PSA glycoforms may be selected from the group consisting of compounds 18, 7, 9, 11, 13, 16, 18, 21 and 25 in Table 1A and B.

In particular embodiments, the at least one di-antennary PSA glycoform is a di-antennary PSA glycoform comprising alpha 2,6-linked sialylation and fucosylation. Such di-antennary, alpha 2,6-linked sialylated and fucosylated PSA glycoforms may be selected from the group consisting of compounds 18, 7, 9, 11, 21 and 25 in Table 1A and B. In embodiments, such di-antennary, alpha 2,6-linked sialylated and fucosylated PSA glycoforms may be selected from the group consisting of compounds 18, 7 and 9 in Table 1A and B.

In particular embodiments, the at least one di-antennary PSA glycoform is a di-antennary PSA glycoform comprising alpha 2,3-linked sialylation. Such di-antennary and alpha 2,3-linked sialylated PSA glycoforms may be selected from the group consisting of compounds 10, 13, 16, 18, 21, 24 and 25 in Table 1A and B. In particular embodiments, the at least one di-antennary PSA glycoform is a di-antennary PSA glycoform comprising alpha 2,6-linked sialylation and alpha 2,3-linked sialylation. Such di-antennary, alpha 2,6-linked sialylated and alpha 2,3-linked sialylated PSA glycoforms may be selected from the group consisting of compounds 13, 16, and 21 in Table 1A and B.

In a particular embodiment the method of the invention can determine whether a subject has aggressive PCa if the level of at least one mono-antennary PSA glycoform is reduced and the level of at least one di-antennary PSA glycoform is increased in the subject's samples when compared to suitable control or reference levels or values.

It will be appreciated that different test samples will likely comprise different amounts of PSA and so the absolute level of any PSA-glycan species detected in a sample will need to be normalised. Normalisation is a standard approach in diagnostic testing. The level of the measured PSA-glycan species can be normalised against the amount of PSA in the original sample. Alternatively, the normalisation can be built into the test assay by spiking the sample to be analysed with a known amount of a reference analyte or marker. For example, the examples herein determined the PSA-glycan levels by reference to a known amount of a particular peptide added/spiked into the protease-digested (e.g. with trypsin or Arg-C (clostripain)) sample. Thus, in a particular embodiment, to normalise the level the sample analysed by MS may be spiked with a known amount of reference PSA peptide derivative (spike peptide). Suitably, the spike peptide may be one that differs from the native PSA peptide (released during the protease-digestion) by a single amino acid and so is classed as a PSA-peptide derivative, MS quantification of the known amount of PSA-peptide derivative alongside quantification of the endogenous peptide allows normalisation of the level of any and each measured PSA-glycan species in the test/case sample. In an embodiment, the spiked peptide may comprise a sequence as disclosed in SEQ ID NO: 2. In another embodiment, the spiked peptide may be labelled, e.g. radiolabelled. Various approaches for normalisation are known to the person skilled in the art.

In particular embodiments of the invention, the detected levels of each PSA-glycan species is normalised. In particular embodiments, the normalised level of a or each PSA-glycan species is determined and used to determine the aggressiveness score.

If a ratio or other equation involving the levels of multiple PSA glycoforms are used then normalization may not be required.

In particular embodiments of the invention, the reference value for the aggressiveness score has been determined from at least one reference population comprising aggressive PCa subjects and/or subjects that do not have aggressive PCa, such as patients with prostatitis, BPH or indolent PCa.

In particular embodiments of the invention, if the level of the at least one mono-antennary PSA glycoform is reduced and the level of the at least one di-antennary PSA glycoform is increased compared to control or reference values the subject is determined to have an aggressive form of PCa. If not, the subject is determined to not have an aggressive form of PCa or to have indolent prostatic disease.

In particular embodiments of the invention, the aggressiveness score for the subject is calculated from a ratio of the level of each measured PSA glycoform determined from the sample.

In particular embodiments of the invention, the method comprises calculating a score for assessing whether the subject has aggressive PCa by taking into consideration the combined level of each determined PSA glycoform.

In particular embodiments of the invention, the method comprises calculating a score (aggressiveness score) for assessing whether the subject has aggressive PCa by taking into consideration the individual levels of the at least one mono-antennary PSA glycoform and the at least one di-antennary PSA glycoform.

In one particular embodiment, the comparison of the individual levels of the at least one mono-antennary PSA glycoform and the at least one di-antennary PSA glycoform to appropriate reference levels is used to aid in determining whether the subject has an aggressive form of PCa or not.

In one particular embodiment, the comparison of the combined levels of the at least one mono-antennary PSA glycoform and the at least one di-antennary PSA glycoform compared to reference level for said combined PSA glycoforms is used to aid in determining whether the subject has an aggressive form of PCa or not.

In one particular embodiment, the aggressiveness score is used to aid in determining whether the subject has an aggressive form of PCa or not.

In one particular embodiment, the score in step (b) comprises or is generated from the ratio of the at least one mono-antennary PSA glycoform and the at least one di-antennary PSA glycoform, or vice versa.

In a particular embodiment, a ratio value comprising the ratio of (i) to (ii), or vice versa, is prepared, wherein (i) is the normalised level of the at least one mono-antennary PSA glycoform in the sample, and (ii) is the normalised levels of the at least one di-antennary PSA glycoform in the sample, optionally, the ratio value is used for calculating a risk score for assessing whether the subject has aggressive PCa, and wherein the ratio value or risk score are then compared to control or reference values to determine whether the subject has an aggressive form of PCa or not.

The method can be applied using measurements from one or multiple mono-antennary species, and one or multiple di-antennary species in the test sample.

In a particular embodiment, the levels of all the measured mono-antennary species are summed up (added together), and the levels of all the measured di-antennary species are summed up and one summed value is divided by the other to give the ratio or “aggressiveness score”. It will be appreciated that if only one mono-antennary species or one di-antennary species in that category is measured then the level of that species is taken as the summed level. In this way the method can be employed using one mono antennary species and two or more di-antennary species or one di-antennary species and two or more mono-antennary species.

In a particular embodiment of the first aspect of the invention at least 3 PSA glycoforms are determined.

In a particular embodiment of the first aspect of the invention at least 4 PSA-glycoforms are determined.

In a particular embodiment of the first aspect of the invention at least 5 PSA-glycoforms are determined.

In a particular embodiment of the first aspect of the invention at least 6 PSA-glycoforms are determined.

The inventors have discovered that the levels of every mono-antennary PSA glycoform (e.g. compounds, 2, 3, 4 and 5) measured in a biofluid sample (e.g. in urine) decreases in subjects with aggressive PCa. Whereas, the levels of the majority of tested di-antennary PSA glycoforms, including for example compounds 7, 9, 18, 24 (see Table 1A and B or Table 6), increase in subjects with aggressive PCa.

In particular embodiments, the PSA glycoforms used or determined in the methods of the invention comprise PSA glycoforms selected from any one of the combinations shown in Table 7 having an AUC (1×CV) of at least 66.0, preferably having an AUC (1×CV) of at least 68.0.

In particular embodiments, the PSA glycoforms used or determined in the methods of the invention comprise PSA glycoforms selected from a group consisting of:
(i) H4N3F1S_{2,6}1 and H4N4F1S_{2,6}1;
(ii) H4N3F1S_{2,6}1 and H5N4F1S_{2,6}1;
(iii) H4N3S_{2,6}1 and H4N5S_{2,3}1(SO₃)1;
(iv) H4N3S_{2,6}1 and H5N4F1S_{2,3}1S_{2,6}1;
(v) H4N3S_{2,6}1 and H4N4F1S_{2,6}1;
(vi) H4N3F1S_{2,6}1 and H5N4F1S_{2,3}1S_{2,6}1;
(vii) H4N3S_{2,6}1 and H4N5F1S_{2,3}1S_{2,6}1;
(viii) H4N3S_{2,6}1 and H4N5F1S_{2,6}1; and
(ix) H3N4F1S_{2,6}1 and H4N5F1S_{2,3}1S_{2,6}1.

As can be seen from Table 1B, the H4N3S_{2,6}1, H4N3F1S_{2,6}1, H3N4F1S_{2,6}1 (underlined directly above) are the mono-antennary compounds 2, 3 and 5.

In a particular embodiment of the first aspect of the invention the levels of at least two PSA glycoforms, at least one a mono-antennary PSA-glycan species and at least one a di-antennary PSA-glycan species, are determined or measured. In a particular embodiment of the first aspect of the invention the levels of just two PSA glycoforms, one a mono-antennary PSA-glycan species and the other a di-antennary PSA-glycan species, are determined or measured. In particular embodiments, the two PSA glycoforms measured are those identified in any one of groups (i) to (ix) immediately above or (a) to (i) immediately below.

In other particular embodiments, the PSA glycoforms used or determined in the methods of the invention comprise PSA glycoforms selected from a group consisting of:
(a) H4N4F1S_{2,6}1 and combination of mono antennary glycoforms;
(b) H4N3S_{2,6}1 and combination of mono antennary glycoforms;
(c) H4N3S_{2,6}1 and combination of a2.3.sialylation glycoforms;
(d) H4N3S_{2,6}1 and combination of a2.6.sialylation glycoforms;
(e) H5N4F1S_{2,3}1S_{2,6}1 and combination of mono antennary glycoforms;
(f) H5N4F1S_{2,3}1 and combination of mono antennary glycoforms;
(g) Combination of a2.3.sialylation glycoforms and combination of mono antennary glycoforms;
(h) H4N5F1S_{2,3}1S_{2,6}1 and combination of mono antennary glycoforms; and
(i) H4N3F1S_{2,3}1 and combination of mono antennary glycoforms.

In particular embodiments, in each of (a) to (i) above, the combination of a class of glycan structure (e.g. combination of mono antennary glycoforms) comprises at least two members, such as 2, 3, 4 or more members from the same class of glycan, e.g. as shown in Table 1A and B.

In other particular embodiments, in each of (a) to (i) above, the combination of a class of glycan structure (e.g. combination of mono antennary glycoforms) comprises all members in that class as shown in Table 1A and B.

Suitable mono-antennary PSA glycoforms for use in the methods of the invention are selected from compounds 2, 3, 4 and 5, as identified in Table 1A and B. Suitable, di-antennary PSA glycoforms for use in the methods of the invention are selected from one or more of compounds 7, 9, 10, 11, 13, 16, 18, 21, 24 and 25, as identified in Table 1A and B.

In particular embodiments of the first aspect of the invention, the levels of 3, 4, 5, or more PSA glycoforms are determined or measured.

In particular embodiments, the PSA glycoforms used or determined in the methods of the invention comprise PSA glycoforms selected from any one of the combinations shown in Table 8 having an AUC (1×CV) of at least 70.0, preferably having an AUC (1×CV) of at least 72.0.

In particular embodiments, the PSA glycoform used or determined in the methods of the invention comprise PSA glycoform selected from any one of the combinations shown in Table 9 having an AUC (1×CV) of at least 70.0, preferably having an AUC (1×CV) of at least 72.0.

In particular embodiments, the PSA glycoform used or determined in the methods of the invention comprise combinations of PSA glycoforms selected from any one of the combinations shown in Table 10 having an AUC (1×CV) of at least 73.0, preferably having an AUC (1×CV) of at least 72.5.

In particular embodiments, the combinations of PSA glycoforms used are a combination of mono-antennary PSA glycoforms selected from at least two of compounds 2, 3, 4 and 5 and a combination of di-antennary PSA glycoforms selected from at least two of compounds 7, 9, 18 and 24.

In particular embodiments, the combinations of PSA glycoforms used are selected from:
(a) Compounds 2, 3 and 5 (as mono-antennary species) and compounds 24, 7 and 9 (as di-antennary species);
(b) Compounds 2 and 3 (as mono-antennary species) and compounds 7 and 9 (as di-antennary species);
(c) Compounds 3 and 5 (as mono-antennary species) and compounds 24, 7 and 9 (as di-antennary species);
(d) Compounds 2, 3 and 5 (as mono-antennary species) and compounds 7 and 9 (as di-antennary species);
(e) Compounds 2 and 3 (as mono-antennary species) and compound 7 (as the di-antennary species); and
(f) Compounds 3 and 5 (as mono-antennary species) and compounds 24 and 9 (as di-antennary species).

In particular embodiments, when the method employs measurements of the level of two PSA-glycan species, the PSA-glycan species measured/used consist of one PSA-glycan species whose level is higher (+ direction) in the samples of subjects with aggressive PCa relative to samples of subjects with indolent prostate disease and one PSA-glycan species whose level are lower (− direction) in the samples of subjects with aggressive PCa relative to samples of subjects with indolent prostate disease. Suitable combinations of two PSA-glycan species for use in the methods of the present invention are any of those shown in Table 7.

In particular embodiments, when the method employs measurements of level of three PSA glycoforms, the PSA-glycoforms measured/used consist of two PSA glycoforms (PSA-glycan species) whose levels increase (+ direction) in the samples of a subject with aggressive PCa and one PSA-glycan species (PSA glycoform) whose level decreases (− direction) in the samples of a subject with aggressive PCa. Suitable combinations of three PSA glycoforms for use in the methods of the present invention are any of those shown in Table 8.

In particular embodiments, when the method employs measurements of level of four PSA glycoforms, the PSA glycoform measured/used consist of three PSA-glycan species whose levels increase (+ direction) in the samples of a subject with aggressive PCa and one PSA glycoform whose level decreases (− direction) in the samples of a subject with aggressive PCa. Suitable combinations of four PSA glycoforms for use in the methods of the present invention are any of those shown in Table 9.

In particular embodiments of any aspect of the invention, the aggressiveness score is calculated by or comprises (i) weighted calculation or (ii) ratio of levels of the PSA glycoform(s).

In a particular embodiment, a calculation based on or comprising a ratio of levels of the PSA glycoform species is performed, wherein the levels of at least one PSA glycoform known to increase in subjects with aggressive PCa and at least one PSA glycoform known to decrease in subjects with aggressive PCa are used. Suitably, the PSA glycoform known to decrease in subjects with aggressive PCa is a mono-antennary PSA glycoform. Suitably, the PSA glycoform known to increase in subjects with aggressive PCa is a di-antennary PSA glycoform.

In particular embodiments, the methods of the invention employ a combination of PSA glycoforms of one glycan class and a combination of PSA glycoform of another glycan class. Suitably, one glycan class comprises the mono-antennary PSA glycoform species. Suitably, one glycan class comprises the di-antennary PSA glycoform species, or a sub-set thereof. By subset thereof we mean the di-antennary species also has one or more specific types of glycosylation (such as sialylation, fucosylation etc.).

In a particular embodiment, the aggressiveness score calculation comprises or consists of forming a ratio of sum level of a combination of PSA glycoform species from one PSA glycoform class over sum level of a combination of PSA glycoform species from a different PSA glycoform class. In a particular embodiment, one of the classes of PSA glycoform species is the mono-antennary class of PSA glycoform species. In a particular embodiment, one of the classes of PSA glycoform species is the di-antennary class of PSA glycoform species.

In a particular embodiment, the aggressiveness score is calculated by ratio of sum level of a combination of mono-antennary PSA glycoform species and the sum level of a combination of di-antennary PSA glycoform species. Particular examples of combinations are provided in Table 10 herein below.

In a particular embodiment, the aggressiveness score is calculated by ratio of sum level of a combination of mono-PSA glycoform species and the sum level of a combination of di-antennary PSA glycoform species that has fucosylation.

In a particular embodiment, the aggressiveness score is calculated by ratio of sum level of a combination of mono-PSA glycoform species and the sum level of a combination of di-antennary PSA glycoform species that has α2, 3-sialylation.

In a particular embodiment, the aggressiveness score is calculated by ratio of sum level of a combination of mono-PSA glycoform species and the sum level of a combination of di-antennary PSA glycoform species that has α2, 6-sialylation.

The inventors have found that determination of aggressiveness based on a ratio of levels of the PSA glycoform species was particularly effective, indeed it yielded improvement of performance in assessing aggressiveness (see Table 10).

One advantage of calculating the aggressiveness score from a ratio of levels of the PSA glycoform species is that no normalization of the levels is required (as normalization will be eliminated by ratio formation.

The methods of the invention can be employed on any suitable subject. Such subject may not have had any prior assessment of their prostate gland. More typically, such subject may have has a digital rectal examination (DRE) and or PSA test. The methods of the present invention are particularly suited for analysing samples from subjects who have free PSA serum levels in the grey zone (2-10 ng/mL). The methods of the present invention are particularly suited for analysing samples from subjects with an enlarged prostate gland (prostate gland enlargement).

Thus, in a particular embodiment, the subject has prostate gland enlargement.

Suitably, prostate gland enlargement has been predetermined, e.g. from DRE or imaging.

In a particular embodiment the subject has been preselected based on having prostate gland enlargement.

In a particular embodiment the subject has been preselected based on total serum PSA levels.

In a particular embodiment the subject has 2-10 ng/mL total PSA serum level.

In a particular embodiment the subject has been preselected based on having 2-10 ng/mL total PSA serum level.

In a particular embodiment, the subject has been preselected based on the levels of percent free serum PSA.

In a particular embodiment, the subject has been preselected based on the percent free serum PSA level being less than or equal to 25%, such as between about 0% and 20%.

In a particular embodiment, the subject has a percent free serum PSA level of less than or equal to 25%, such as between about 0% and 20%.

The methods of the invention are based on measurements of PSA-glycan species levels in a sample of a subject. In particular embodiments, the sample is a biofluid sample, such as a subject's body fluid. In particular embodiments, the biofluid sample is selected from: blood, serum, plasma, seminal fluid, prostatic fluid and urine.

In one embodiment, the sample is a urine sample, such as a digital rectal examination (DRE) urine sample.

In particular embodiments, the PSA protein in the sample is processed to separate the PSA protein from other components in the sample.

In particular embodiments, the PSA protein in the sample is isolated or purified from the sample. This ensures that any glycan species subsequently detected are ones that are associated with the PSA in the sample. In a particular embodiment, the PSA protein in the sample is isolated or purified from the sample prior to determining the levels of the PSA-glycan species.

In a particular embodiment, the PSA protein in the subject's biofluid sample is separated from other components in the sample. In a particular embodiment, the PSA protein in the subject's biofluid sample is separated from other components in sample prior to determining the levels of the PSA-glycan species.

As used herein, the term “separated”, “isolated” and “purified” can be used synonymously.

The PSA can be separated from the sample using various conventional purification methods, such as affinity chromatography. Most suitably, the PSA is isolated or purified from the sample by use of a PSA binding partner, such as an antibody that can capture the protein, e.g. PSA binding partner/antibody may be bound to a solid surface such as a microtiter plate or bead and once the PSA is bound to the binding partner the sample can be washed away leaving the PSA protein bound to the immobilised PSA binding partner/antibody. The sample separated/purified PSA can then be disassociated from the binding partner.

In particular embodiments, the biofluid sample is treated to isolate the PSA protein from the sample.

In particular embodiments, the PSA in the sample is isolated from the sample by use of a capture binding partner, such as an anti-PSA antibody. Suitably, the capture-binding partner is attached to a solid surface, such as a bead.

The particular glycan species attached to PSA need to be detected and quantified. This can be done using a variety of techniques. Most suitably, the detection and quantification is done by mass spectrometry conducted after a means of separating the various analytes, such as by using capillary electrophoresis. To facilitate detection by mass spectrometry it is convenient to release the N-glycans from the PSA and analyse N-glycans separately, or digest the PSA (with a suitable protease such as trypsin) to create smaller molecules (with intact glycan species) that are more amenable to MS analysis, and analyse the N-glycopeptides.

Thus, in particular embodiments, the PSA in the sample or that isolated from the sample is treated to release the glycan structures from PSA.

PSA is N-glycosylated at the asparagine residue at position 69 of the PSA protein (Kabat numbering). This location is conveniently situated adjacent to a trypsin cleavage site. Accordingly, the glycan structure(s) attached to PSA can be released as glycopeptide species (different glycoforms) by treating the PSA with a suitable protease, such as trypsin or Arg-C (clostripain).

In a particular embodiment, the PSA protein once separated from the sample is treated with a protease to produce N₆₉ glycopeptide forms.

In a particular embodiment, the PSA protein once separated from the sample is treated with trypsin to produce N₆₉K di-peptide glycoforms.

In a particular embodiment, the PSA protein once separated from the sample is treated with Arg-C (clostripain) to produce N₆₉KSVILLGR glycopeptide forms. The sequence of the NKSVILLGR peptide is identified herein as SEQ ID NO: 3.

The glycan species or its fragments can be released from the purified PSA or the N₆₉ glycopeptides produced as described above, by treatment with a glycosidases, such as one selected from the group consisting of: PNGase F, endoglycosidase F1, endoglycosidase F2, endoglycosidase F3, and endoglycosidase H.

Thus, in particular embodiments, the separated/purified PSA or N₆₉ glycopeptide(s) are treated with a glycosidase to release the glycoform from the PSA protein or N₆₉ glycopeptide (e.g. N₆₉K or N₆₉KSVILLGR glycopeptide).

In particular embodiments, the level of each measured PSA glycoform is determined by mass spectrometry, such as CE-ESI-MS or LC-ESI-MS(/MS), or MALDI-TOF MS A CE-ESI-MS technique suitable for use in the methods of the invention is described in Kammeijer et al (supra).

In particular embodiments, the particular PSA glycoform is detected as its N₆₉K di-peptide glycoform.

In particular embodiments, the particular PSA glycoform is detected as its Ne-KSVILLGR glycopeptide.

The methods of the invention provide valuable information about the status of prostatic disease in a subject. The ability to more accurately determine whether a subject is likely to have an aggressive form of PCa or indolent prostatic disease provides the attending physician and the subject with additional options for further diagnosis or treatment. For example, subjects which are determined to have indolent prostatic disease can be managed by watchful waiting. Subjects which are determined to have or be likely to have aggressive PCa can be identified or selected for biopsy testing and or PCa treatment (such as surgery, radiotherapy, chemotherapy, androgen therapy, and the like).

Thus, in particular embodiments, the determining by the methods of the invention is used to provide a treatment recommendation for the subject and/or to decide whether a prostate biopsy is needed.

It will be appreciated that the methods can be carried out remotely from the subject or their attending physician, indeed, they could be carried out off-shore and the results communicated back.

In particular embodiments, the results of the methods of the invention (e.g. determining whether or not a subject has aggressive form of PCa) are provided to a third party, such as the subject or their attending physician, a laboratory or health centre.

In particular embodiments, in the method of the invention the aggressiveness score is obtained from an algorithm into which the levels of the PSA glycoforms have been entered.

In particular embodiments, the algorithm has been generated by machine learning trained on samples from subjects with aggressive PCa and subjects with indolent PCa and/or BPH and/or healthy subjects.

The methods of the invention may involve analysing a sample from a subject to determine the level at least one PSA glycoform and then interpreting the data to determine whether or not the subject has or is likely to have aggressive form of PCa. However, it is possible that the level(s) of the one or more PSA glycoform(s)has already been established but the interrogation or analysis or the data has not been undertaken so the status of the subject is not determined. The data of the level at the least one PSA glycoform can be input into a computer, e.g. via a computer program, to conduct the analysis. Suitably, the computer comprises software with algorithms and reference values that can deliver the determination of the status of the subject.

According to a second aspect of the invention there is provided a method for determining whether or not a subject has an aggressive form of prostate cancer, comprising:
(i) isolating PSA protein from other proteins in a biofluid sample from the subject;
(ii) treating the isolated PSA protein with a protease;
(iii) determining the level of each of two or more N₆₉ glycopeptide forms in the sample, wherein at least one of the N₆₉ glycopeptide forms is a mono-antennary glycoform and at least one of the N₆₉ glycopeptide forms is a di-antennary glycoform;
(iv) determining an aggressiveness score for the subject using the levels of each Nee glycopeptide forms in (iii);
(v) comparing the aggressiveness score score in (iv) to a reference value for said aggressiveness score; and
(vi) based on the comparison in step (v) determining whether the subject has an aggressive form of PCa or not.

This second aspect of the invention is a subset of the first aspect of the invention but wherein the sample from the subject is treated to isolate the PSA, cleaved with a protease (such as trypsin or Arg-C (clostripain)), and the levels of the N₆₉ glycopeptides are determined and assessed. As such, the embodiments set out for the first aspect of the invention also apply to this second aspect of the invention unless it is apparent from the context that it does not apply.

In a particular embodiment, the protease is capable of releasing the N₆₉ comprising glycopeptides. In particular embodiments, the protease is trypsin or Arg-C (clostripain).

In particular embodiments, the first mono-antennary PSA glycoform of step (a) is selected from compound 2, 3, 4 or 5 (as shown in Tables 1A and B).

In particular embodiments, the first di-antennary PSA glycoform of step (b) is selected from compound 7, 9, 10, 11, 13, 16, 18, 21, 24 or 25, (as shown in Tables 1A and B).

The first, second and fourth aspects of the invention involve measurements of the levels of at least 2 PSA glycoforms. However, as can be seen from Table 2, the levels of three PSA glycoforms(compounds 21, 2 and 9) when analysed individually are capable of discriminating aggressive from indolent PCa and BPH with a greater specificity (higher AUC) than free PSA. These PSA glycoforms can therefore be used individually in a method to aid in determining whether the subject has aggressive PCa or not.

Thus, according to a third aspect of the invention there is provided a method to aid in determining whether or not a subject has an aggressive form of prostate cancer, comprising:
(a) determining the level of a PSA glycoform selected from the group consisting of: H4N5F1S_{2,3}1S_{2,6}1; H4N3S_{2,6}1; and H5N₄F1S_{2,6}1 in a biofluid sample from the subject,
(b) comparing the value for the level of the PSA glycoform in (a) to a reference value for said PSA glycoform; or comparing an aggressiveness score taking into account the level said PSA glycoform in (a) to a reference value for said aggressiveness score; and
(c) using the comparison in (b) to aid in determining whether the subject has an aggressive form of prostate cancer or not.

In a particular embodiment, the reference value is a value correlating with aggressive PCa. In a particular embodiment, the reference value is a value correlating with non-aggressive PCa, such as BPH.

As appropriate the embodiments applicable to the first aspect of the invention can be applied to this third aspect of the invention.

In a particular embodiment of the third aspect of the invention the method relies on measurement of the level of H4N5F1S_{2,3}1S_{2,6}1 (compound 21). In a particular embodiment, H4N5F1S_{2,3}1S_{2,6}1 (compound 21) is the only measured PSA glycoform. Levels of this PSA glycoform are increased in subjects with aggressive PCa compared to those with indolent prostatic disease. Thus, in a particular embodiment, if the level of H4N5F1S_{2,3}1S_{2,6}1 is increased relative to a reference value the subject is determined to have an aggressive form of PCa. In another embodiment, if the level of H4N5F1S_{2,3}1S_{2,6}1 is not increased relative to a reference value the subject is determined to not have an aggressive form of PCa and/or determined to have indolent prostatic disease.

In a particular embodiment of the third aspect of the invention, the method relies on measurement of the level of H4N3S_{2,6}1 (compound 2). In a particular embodiment, H4N3S_{2,6}1 (compound 2) is the only measured PSA glycoform. Levels of this PSA glycoform are decreased in subjects with aggressive PCa than those with indolent prostatic disease. Thus, in a particular embodiment, if the level of H4N3S_{2,6}1 is decreased relative to a reference value the subject is determined to have an aggressive form of PCa. In another embodiment, if the level of H4N3S_{2,6}1 is not decreased relative to a reference value the subject is determined to have not have an aggressive form of PCa and/or determined to have indolent prostatic disease.

In a particular embodiment of the third aspect of the invention the method relies on measurement of the level of H5N4F1S_{2,6}1 (compound 9). In a particular embodiment, H5N4F1S_{2,6}1 (compound 9) is the only measured PSA glycoform. Levels of this PSA glycoform are increased in subjects with aggressive PCa than those with indolent prostatic disease.

Thus, in a particular embodiment, if the level of H5N4F1S_{2,6}1 is increased relative to a reference value the subject is determined to have an aggressive form of PCa.

In another embodiment, if the level of H5N4F1S_{2,6}1 is not increased relative to a reference value the subject is determined to have not have an aggressive form of PCa and/or determined to have indolent prostatic disease.

In particular embodiments of the invention, the detected levels of each PSA-glycan is normalised. In particular embodiments, the normalised level of a or each PSA-glycan is determined and/or compared to the control or reference value.

In particular embodiments of the invention, the reference value has been determined from at least one reference population comprising aggressive PCa subjects and subjects that do not have aggressive PCa, such as patients with prostatitis, BPH or indolent PCa.

In particular embodiments of the invention, the method comprises calculating a score for assessing whether the subject has aggressive PCa by taking into consideration the individual levels of the at least one PSA glycoform.

In one particular embodiment, the score calculated by taking into consideration the level of the individual PSA glycoform is used to aid in determining whether the subject has an aggressive form of PCa or not.

In a particular embodiment, the subject has prostate gland enlargement. Suitably, prostate gland enlargement has been predetermined, e.g. from DRE or imaging.

In a particular embodiment the subject has been preselected based on total serum PSA levels.

In a particular embodiment the subject has about 2-10 ng/mL total PSA serum level.

In a particular embodiment the subject has been preselected based on having about 2-10 ng/mL total PSA serum level.

In a particular embodiment, the subject has been preselected based on the levels of percent free serum PSA.

In a particular embodiment, the subject has been preselected based on the percent free serum PSA level being less than or equal to 25%, such as between about 0% and 20%.

In a particular embodiment, the subject has a percent free serum PSA level of less than or equal to 25%, such as between about 0% and 20%.

The particular embodiments, the sample is any bodily fluid that contains PSA. In particular embodiments, the sample is a biofluid sample. In particular embodiments, the biofluid sample is a body fluid. In particular embodiments, the sample is selected from: blood, serum, plasma, seminal fluid, prostatic fluid and urine.

In one embodiment, the sample is a urine sample, such as a digital rectal examination (DRE) urine sample.

In particular embodiments, the PSA protein in the sample is isolated or purified from the sample. This ensures that any glycan species subsequently detected are ones that are associated with the PSA in the sample.

In particular embodiments, the PSA protein in the subject's biofluid sample is separated from other components in the sample.

The PSA can be separated from the sample using various conventional purification methods, such as affinity chromatography. Most suitably, the PSA is isolated or purified from the sample by use of a PSA binding partner, such as an antibody that can capture the protein, e.g. PSA binding partner/antibody may be bound to a solid surface such as a microtiter plate or bead and once the PSA is bound to the binding partner the sample can be washed away leaving the PSA protein bound to the immobilised PSA binding partner/antibody. The sample separated/purified PSA can then be disassociated from the binding partner.

In particular embodiments, the biofluid sample is treated to isolate the PSA protein from the sample.

In particular embodiments, the PSA in the sample is isolated from the sample by use of a capture binding partner, such as an anti-PSA antibody. Suitably, the capture-binding partner is attached to a solid surface, such as a bead.

The particular glycan attached to PSA need to be detected and quantified. This can be done using a variety of techniques.

Most suitably, the detection and quantification is done by mass spectrometry conducted after a means of separating the various analytes, such as by using capillary electrophoresis.

To facilitate detection my mass spectrometry it is convenient to release the glycan from the PSA or treat the PSA-glycan to create smaller molecules (with intact glycan species) that are more amenable to MS analysis.

Thus, in particular embodiments, the PSA in the sample or that isolated from the sample is treated to release the glycan structures from PSA.

In a particular embodiment, the PSA protein once separated from the sample is treated with trypsin to produce N₆₉K di-peptide glycopeptides (glycoforms).

In a particular embodiment, the PSA protein once separated from the sample is treated with Arg-C (clostripain) to produce N₆₉ comprising glycopeptides.

In particular embodiments, the separated/purified PSA or glycopeptide (e.g. N₆₉ comprising glycopeptide forms) are incubated with a glycosidase to release the glycoform from the PSA protein or glycopeptide.

In particular embodiments, the level of each measured PSA glycoform is determined by mass spectrometry, such as CE-ESI-MS or LC-ESI-MS(/MS), or MALDI-TOF MS

In particular embodiments, the particular PSA glycoform is detected as its N₆₉K di-peptide glycoform or its N₆₉KSVILLGR glycopeptide form.

The methods of the invention provide valuable information about the status of prostatic disease in a subject. The ability to more accurately determine whether a subject is likely to have an aggressive form of PCa or indolent prostatic disease provides the attending physician and the subject with additional options for further diagnosis or treatment. For example, subjects which are determined to have indolent prostatic disease can be managed by watchful waiting. Subjects which are determined to have or be likely to have aggressive PCa can be identified or selected for biopsy testing and or PCa treatment (such as surgery, radiotherapy, chemotherapy, androgen therapy, and the like).

Thus, in particular embodiments, the determining by the methods of the invention is used to provide a treatment recommendation for the subject and/or to decide whether a prostate biopsy is needed.

In particular embodiments, in the method of the invention an algorithm is applied to the levels PSA glycoforms to determine whether the subject has an aggressive form of PCa or not.

In particular embodiments, the algorithm has been generated by machine learning trained on samples from subjects with aggressive PCa and subjects with indolent PCa and/or BPH and/or healthy subjects.

The methods of the invention may involve analysing a sample from a subject to determine the level at least one PSA glycoform and then interpreting the data to determine whether or not the subject has or is likely to have aggressive form of PCa. However, it is possible that the level(s) of the one or more PSA glycoform has already been established but the interrogation or analysis or the data has not been undertaken so the status of the subject is not determined. The data of the level at the least one PSA glycoform can be input into a computer to conduct the analysis. Suitably, the computer comprises software with algorithms and reference values that can deliver the determination of the status of the subject.

### Computer-Implemented Methods

According to a fourth aspect of the invention there is provided a computer-implemented method to aid in determining whether a subject has an aggressive form of prostate cancer or not, comprising the steps of:
(a) receiving a value for the level of a first mono-antennary PSA glycoform in a biofluid sample of the subject;
(b) receiving a value for the level of a first di-antennary PSA glycoform in the biofluid sample of the subject;
(c) calculating an aggressiveness score for the subject based on the levels received in (a) and (b);
(d) comparing the subject's aggressiveness score in (c) to a reference value for said aggressiveness score; and
(e) using the comparison in (d) to aid in determining whether the subject has an aggressive form of prostate cancer or not.

In particular embodiments, the first mono-antennary PSA glycoform of step (a) is selected from compound 2, 3, 4 or 5 (as shown in Tables 1A and B).

In particular embodiments, the first di-antennary PSA glycoform of step (b) is selected from compound 7, 9, 10, 11, 13, 16, 18, 21, 24 or 25, (as shown in Tables 1A and B).

In particular embodiments, the PSA glycoform used or determined in the fourth aspect of the invention comprise PSA glycoform selected from any one of the combinations shown in Table 8 having an AUC (1×CV) of at least 70.0, preferably having an AUC (1×CV) of at least 72.0.

In particular embodiments, the PSA glycoform used or determined in the fourth aspect of comprise PSA glycoform selected from any one of the combinations shown in Table 9 having an AUC (1×CV) of at least 70.0, preferably having an AUC (1×CV) of at least 72.0.

In particular embodiments, the PSA glycoform used or determined in this aspect of the invention comprise combinations of PSA glycoforms selected from any one of the combinations shown in Table 10 having an AUC (1×CV) of at least 73.0, preferably having an AUC (1×CV) of at least 72.5.

In particular embodiments, the combinations of PSA glycoforms used are a combination of mono-antennary PSA glycoforms selected from at least two of compounds 2, 3, 4 and 5 and a combination of di-antennary PSA glycoforms selected from at least two of compounds 7, 9, 18 and 24.

In particular embodiments, the combinations of PSA glycoforms used are selected from:
(a) Compounds 2, 3 and 5 (as mono-antennary species) and compounds 24, 7 and 9 as di-antennary species.
(b) Compounds 2 and 3 (as mono-antennary species) and compounds 7 and 9 as di-antennary species.
(c) Compounds 3 and 5 (as mono-antennary species) and compounds 24, 7 and 9 as di-antennary species.
(d) Compounds 2, 3 and 5 (as mono-antennary species) and compounds 7 and 9 as di-antennary species.
(e) Compounds 2 and 3 (as mono-antennary species) and compound 7 and as the di-antennary species.
(f) Compounds 3 and 5 (as mono-antennary species) and compounds 24 and 9 as di-antennary species.

As appropriate, the embodiments applicable to the first aspect of the invention can be applied to this fourth aspect of the invention.

According to a fifth aspect of the invention there is provided a computer-implemented method to aid in determining whether a subject has an aggressive form of prostate PCa or not, comprising the steps of:
(a) receiving a value for the level of a PSA glycoform selected from the group consisting of H4N5F1S_{2,3}1S_{2,6}1; H4N3S_{2,6}1; and H5N₄F1S_{2,6}1 in a biofluid sample of the subject;
(b) comparing the value for the level of the PSA glycoform received in (a) to a reference value for said PSA glycoform; or comparing an aggressiveness score taking into account the level of the PSA glycoform received in (a) to a reference value for said aggressiveness score; and
(c) using the comparison in (b) to aid in determining whether or not the subject has an aggressive form of prostate cancer.

The values received in (a) can be obtained from the PSA glycoform determined in the third aspect of the invention.

According to a variation of this fifth aspect of the invention there is provided a computer-implemented method to aid in determining whether a subject has an aggressive form of prostate cancer or not, comprising the steps of:
(a) receiving a value for the level of a PSA glycoform selected from the group consisting of H4N5F1S_{2,3}1S_{2,6}1; H4N3S_{2,6}1; and H5N4F1S_{2,6}1 in a biofluid sample of the subject;
(b) calculating an aggressiveness score for the subject taking into account the value received in (a);
(c) comparing the aggressiveness score in (b) to a reference aggressiveness score; and
(d) using the comparison in (c) to aid in determining whether or not the subject has an aggressive form of prostate cancer.

As appropriate the embodiments applicable to the third aspect of the invention can be applied to this fifth aspect of the invention.

The term “computer-implemented” as used herein means that the method is carried out in an automated fashion on a data processing unit which is, typically, comprised in a computer or similar data processing device. The data processing unit shall receive values for the level of the biomarkers (i.e. level of each PSA glycoform). Such values can be the amounts, relative amounts or any other calculated value reflecting the amount as described elsewhere herein in detail. Accordingly, it is to be understood that the aforementioned method does not require the determination of amounts for the biomarkers but rather uses values for already predetermined amounts.

The present invention also, in principle, contemplates a computer program, computer program product or computer readable storage medium having tangibly embedded said computer program, wherein the computer program comprises instructions which, when run on a data processing device or computer, carry out the method of the present invention as specified above. Specifically, the present disclosure further encompasses:
a computer or computer network comprising at least one processor, wherein the processor is adapted to perform the method according to one of the aspects described in this description,
a computer loadable data structure that is adapted to perform the method according to one of the aspects described in this description while the data structure is being executed on a computer,
a computer script, wherein the computer program is adapted to perform the method according to one of the aspects described in this description while the program is being executed on a computer,
a computer program comprising program means for performing the method according to one of the aspects described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the aspects described in this description after having been loaded into a main and/or working storage of a computer or of a computer network,
a computer program product having program code means, wherein the program code means can be stored or are stored on a storage medium, for performing the method according to one of the aspects described in this description, if the program code means are executed on a computer or on a computer network,
a data stream signal, typically encrypted, comprising a data for parameters as defined herein elsewhere, and
a data stream signal, typically encrypted, comprising the assessment provided by the methods of the present invention.

The method of the first, second, third, fourth and fifth aspects of the invention can be used to determine or to aid in determining whether a subject has an aggressive form of PCa or to determine or to aid in determining whether a subject does not have an aggressive form of PCa, such as indolent prostatic disease. Suitably, the method of the first, second, third, fourth or fifth aspects of the invention can be used to determine or to aid in determining whether the subject has indolent disease, such as BPH.

By “aid in determining” we mean that other factors may or may not be used in addition to make the determination and so the measure that is the “aid” is only part of the determination.

As used herein if an outcome can be determined directly, e.g. from the aggressiveness score, this can be considered to be an aid in determining.

### Kit of Parts

According to the sixth aspect of the invention there is provided a kit for use (or use of a kit) in a method of the invention (including any one of the first, second, third, fourth or fifth aspects of the invention), the kit comprising a scoring system with PSA glycoform threshold values or ratios indicative of aggressive PCa.

Optionally, the kit also comprises instructions for use.

Optionally the kit also comprises a PSA binding partner, such as an antibody, in particular a monoclonal antibody, optionally wherein the PSA binding partner is immobilised onto a solid surface, such as a bead.

Such a PSA binding partner may be for isolating or purifying PSA protein. In embodiments, the kit may comprise a protease, such as trypsin or Arg C. Trypsin or Arg-C (clostripain) can be used to generate glycopeptides of PSA that can be measured via mass spectrometry. In embodiments, the kit may be a kit for mass spectrometry detection of PSA glycoforms.

### Further Aspects and Embodiments of the Invention

According to an seventh aspect of the invention there is provided a method for determining whether a patient has aggressive prostate cancer or indolent prostatic disease comprising:
(a) determining the level of one or more PSA glycoform(s) in a urine sample from the patient;
(b) calculating an aggressiveness score for the subject taking into consideration the level(s) in (a)
(c) comparing the aggressiveness score of (b) to a reference value of said aggressiveness score; and
(d) based on the comparison in step (b) determining whether the patient has aggressive prostate cancer or indolent prostatic disease.

In particular embodiments, the one or more PSA glycoform(s) are selected from one or more of compounds: 2, 3, 4, 5, 7, 9, 10, 11, 13, 16, 18, 21, 24 and 25, as identified in Table 1A and B. Most suitably, the PSA glycoform is selected from: H4N5F1S_{2,3}1 S_{2,6}1; H4N3S_{2,6}1; and H5N4F1S_{2,6}1. As appropriate the embodiments applicable to the third aspect of the invention can be applied to this seventh aspect of the invention.

According to an eighth aspect of the invention there is provided a method of distinguishing whether a subject likely has aggressive prostate cancer or indolent prostatic disease comprising the steps of:
(a) determining or receiving the level of (i) one or more mono-antennary PSA-glycoform(s) and (ii) one or more di-antennary PSA glycoform(s) in a bio-fluid sample of the subject;
(b) calculating an aggressiveness score using the levels detected in (a);
(c) comparing the aggressiveness score determined in step (b) with a reference value of said aggressiveness score; and
(d) based on the comparison in step (c) determining whether the subject aggressive prostate cancer or indolent prostatic disease.

In a particular embodiment, the levels in step (a) were determined from a bio-fluid sample of the subject.

In particular embodiments, the one or more PSA glycoforms are selected from one or more of compounds: 2, 3, 4, 5, 7, 9, 10, 11, 13, 16, 18, 21, 24 and 25, as identified in Table 1A and B.

In particular embodiments, the PSA glycoforms used or determined in the seventh or eighth aspects of the invention comprise PSA glycoforms selected from any one of the combinations shown in Table 8 having an AUC (1×CV) of at least 70.0, preferably having an AUC (1×CV) of at least 72.0.

In particular embodiments, the PSA glycoforms used or determined in the seventh or eighth aspect of the invention comprise PSA glycoforms selected from any one of the combinations shown in Table 9 having an AUC (1×CV) of at least 70.0, preferably having an AUC (1×CV) of at least 72.0.

In particular embodiments, the PSA glycoforms used or determined in the seventh or eighth aspect of the invention comprise combinations of PSA glycoforms selected from any one of the combinations shown in Table 10 having an AUC (1×CV) of at least 73.0, preferably having an AUC (1×CV) of at least 72.5.

In particular embodiments, the combinations of PSA glycoforms used are a combination of mono-antennary PSA glycoforms selected from at least two of compounds 2, 3, 4 and 5 and a combination of di-antennary PSA glycoforms selected from at least two of compounds 7, 9, 18 and 24.

In particular embodiments, the combinations of PSA glycoforms used are selected from:
(a) Compounds 2, 3 and 5 (as mono-antennary species) and compounds 24, 7 and 9 as di-antennary species.
(b) Compounds 2 and 3 (as mono-antennary species) and compounds 7 and 9 as di-antennary species.
(c) Compounds 3 and 5 (as mono-antennary species) and compounds 24, 7 and 9 as di-antennary species.
(d) Compounds 2, 3 and 5 (as mono-antennary species) and compounds 7 and 9 as di-antennary species.
(e) Compounds 2 and 3 (as mono-antennary species) and compound 7 and as the di-antennary species.
(f) Compounds 3 and 5 (as mono-antennary species) and compounds 24 and 9 as di-antennary species.

As appropriate the embodiments applicable to the first aspect of the invention can be applied to this eighth aspect of the invention.

TABLE 1A
List of PSA-glycoforms, the direction of expression change* in aggressive
PCa subjects and the AUC value for the single marker.

| | Sum formula of | Sum formula of | | | |
|---|---|---|---|---|---|
| | PSA glycoform | PSA glycoform | | | |
| | according to | according to | N-acetyl | | |
| Compound | standard | alternative | neuraminic | Expression | AUC single |
| number | nomenclature | nomenclature | acid linkage | change* | marker |
| 1 | H5N2 | HexNac(2)Hex(5) | — | + | n/a, only in |
| | | | | | multivariate |
| | | | | | setting for |
| | | | | | BPH vs Pca |
| 2 | H4N3S_{2,6}1 | HexNac(3)Hex(4)Neu5Ac(1) | Neu5Ac(a2,6) | − | 63 |
| 3 | H4N3F1S_{2,6}1 | HexNac(3)Hex(4)Fuc(1) | Neu5Ac(a2,6) | − | 59.1 |
| | | Neu5Ac(1) | | | |
| 4 | H4N3F1S_{2,3}1 | HexNac(3)Hex(4)Fuc(1) | Neu5Ac(a2,3) | − | n/a, only in |
| | | Neu5Ac(1) | | | multivariate |
| | | | | | setting or for |
| | | | | | BPH vs. PCa |
| 5 | H3N4F1S_{2,6}1 | HexNac(4)Hex(3)Fuc(1) | Neu5Ac(a2,6) | − | n/a, only in |
| | | Neu5Ac(1) | | | multivariate |
| | | | | | setting |
| 6 | H6N3S_{2,6}1 | HexNac(3)Hex(6)Neu5Ac(1) | Neu5Ac(a2,6) | ns | n/a |
| 7 | H4N4F1S_{2,6}1 | HexNac(4)Hex(4)Fuc(1) | Neu5Ac(a2,6) | + | 59.1 |
| | | Neu5Ac(1) | | | |
| 8 | H5N4S_{2,6}1 | HexNac(4)Hex(5)Neu5Ac(1) | Neu5Ac(a2,6) | ns | n/a |
| 9 | H5N4F1S_{2,6}1 | HexNac(4)Hex(5)Fuc(1) | Neu5Ac(a2,6) | + | 62 |
| | | Neu5Ac(1) | | | |
| 10 | H5N4F1S_{2,3}1 | HexNac(4)Hex(5)Fuc(1) | Neu5Ac(a2,3) | + | 58.1 |
| | | Neu5Ac(1) | | | |
| 11 | H4N5F1S_{2,6}1 | HexNac(5)Hex(4)Fuc(1) | Neu5Ac(a2,6) | + | 60.5 |
| | | Neu5Ac(1) | | | |
| 12 | H5N4S_{2,6}2 | HexNac(4)Hex(5)Neu5Ac(2) | Neu5Ac(a2,6) + | − | 59.9 |
| | | | Neu5Ac(a2,6) | | |
| 13 | H5N4S_{2,3}1S_{2,6}1 | HexNac(4)Hex(5)Neu5Ac(2) | Neu5Ac(a2,3) + | + | n/a, only in |
| | | | Neu5Ac(a2,6) | | multivariate |
| | | | | | setting |
| 14 | H5N4S_{2,3}2 | HexNac(4)Hex(5)Neu5Ac(2) | Neu5Ac(a2,3) + | + | 60.7 |
| | | | Neu5Ac(a2,3) | | |
| 15 | H4N5S_{2,6}2 | HexNac(5)Hex(4)Neu5Ac(2) | Neu5Ac(a2,6) + | + | n/a, only in |
| | | | Neu5Ac(a2,6) | | multivariate |
| | | | | | setting |
| 16 | H4N5S_{2,3}1S_{2,6}1 | HexNac(5)Hex(4)Neu5Ac(2) | Neu5Ac(a2,3) + | + | n/a, only in |
| | | | Neu5Ac(a2,6) | | multivariate |
| | | | | | setting |
| 17 | H5N4F1S_{2,6}2 | HexNac(4)Hex(5)Fuc(1) | Neu5Ac(a2,6) + | + | n/a, only in |
| | | Neu5Ac(2) | Neu5Ac(a2,6) | | multivariate |
| | | | | | setting for |
| | | | | | BPH vs Pca |
| 18 | H5N4F1S_{2,3}1S_{2,6}1 | HexNac(4)Hex(5)Fuc(1) | Neu5Ac(a2,3) + | + | 61.1 |
| | | Neu5Ac(2) | Neu5Ac(a2,6) | | |
| 19 | H5N4F1S_{2,3}2 | HexNac(4)Hex(5)Fuc(1) | Neu5Ac(a2,3) + | + | 60.1 |
| | | Neu5Ac(2) | Neu5Ac(a2,3) | | |
| 20 | H4N5F1S_{2,6}2 | HexNac(5)Hex(4)Fuc(1) | Neu5Ac(a2,6) + | + | 60.3 |
| | | Neu5Ac(2) | Neu5Ac(a2,6) | | |
| 21 | H4N5F1S_{2,3}1S_{2,6}1 | HexNac(5)Hex(4)Fuc(1) | Neu5Ac(a2,3) + | + | 64.1 |
| | | Neu5Ac(2) | Neu5Ac(a2,6) | | |
| 22 | H3N6F1S_{2,6}2 | HexNac(6)Hex(3)Fuc(1) | Neu5Ac(a2,6) + | + | 59.9 |
| | | Neu5Ac(2) | Neu5Ac(a2,6) | | |
| 23 | H4N5S_{2,6}1(SO₃)1 | HexNac(5)Hex(4)Neu5Ac(1) | Neu5Ac(a2,6) | − | n/a, only in |
| | | | | | multivariate |
| | | | | | setting for |
| | | | | | BPH vs Pca |
| 24 | H4N5S_{2,3}1(SO₃)1 | HexNac(5)Hex(4)Neu5Ac(1) | Neu5Ac(a2,3) | + | 59.1 |
| 25 | H4N5F1S_{2,6}1(SO₃)1 | HexNac(5)Hex(4)Fuc(1) | Neu5Ac(a2,6) | − | n/a, only in |
| | | Neu5Ac(1) | | | multivariate |
| | | | | | setting or for |
| | | | | | BPH vs. Pca |
| 26 | H4N5F1S_{2,3}1(SO₃)1 | HexNac(5)Hex(4)Fuc(1) | Neu5Ac(a2,3) | + | 58.5 |
| | | Neu5Ac(1) | | | |

Abbreviations :
H: Hex: Hexose, exemplary Mannose (Man) or Galactose (Gal)
N: HexNAc: N-acetylhexosamine, exemplary N-acetylglucosamine (GlcNAc) or N-acetylgalactosamine (GalNAc)
F: Fuc: Fucose; deoxy hexose
S: Neu5Ac: N-acetyl neuraminic acid, sialic acid
S_{2,6}: Neu5Ac a2-6: alpha 2,6-linked sialic acid
S_{2,3}: Neu5Ac a2-3: alpha 2,3-linked sialic acid
(SO₃): Sulfation
*Expression change reflects the change in expression level direction of the individual (univariate analysis) PSA-glycan in aggressive PCa subjects vis-à-vis indolent prostate disease subjects (indolent PCa or benign hyperplasia), no change = nc; + = up; − = down, ns = not selected.

TABLE 1B
The individual PSA-glycan species, the category or class they belong
to and the mass to charge (m/z) ratio of the N₆₉K dipeptide glycoform

| | | Complex | | alpha2,3sialylation | alpha2,6sialylation | Di-sialylation | Fucosylation |
|---|---|---|---|---|---|---|---|
| Compound | | mono- | Complex | on complex | on complex | on complex | on complex |
| ID | Complex | antennary | diantennary | diantennary | diantennary | diantennary | diantennary |
| 1 | 0 | 0 | 0 | 0 | 0 | 0 | 0 |
| 2 | 1 | 1 | 0 | 0 | 0 | 0 | 0 |
| 3 | 1 | 1 | 0 | 0 | 0 | 0 | 0 |
| 4 | 1 | 1 | 0 | 0 | 0 | 0 | 0 |
| 5 | 1 | 1 | 0 | 0 | 0 | 0 | 0 |
| 6 | 0 | 0 | 0 | 0 | 0 | 0 | 0 |
| 7 | 1 | 0 | 1 | 0 | 1 | 0 | 1 |
| 8 | 1 | 0 | 1 | 0 | 1 | 0 | 0 |
| 9 | 1 | 0 | 1 | 0 | 1 | 0 | 1 |
| 10 | 1 | 0 | 1 | 1 | 0 | 0 | 1 |
| 11 | 1 | 0 | 1 | 0 | 1 | 0 | 1 |
| 12 | 1 | 0 | 1 | 0 | 2 | 1 | 0 |
| 13 | 1 | 0 | 1 | 1 | 1 | 1 | 0 |
| 14 | 1 | 0 | 1 | 2 | 0 | 1 | 0 |
| 15 | 1 | 0 | 1 | 0 | 2 | 1 | 0 |
| 16 | 1 | 0 | 1 | 1 | 1 | 1 | 0 |
| 17 | 1 | 0 | 1 | 0 | 2 | 1 | 1 |
| 18 | 1 | 0 | 1 | 1 | 1 | 1 | 1 |
| 19 | 1 | 0 | 1 | 2 | 0 | 1 | 1 |
| 20 | 1 | 0 | 1 | 0 | 2 | 1 | 1 |
| 21 | 1 | 0 | 1 | 1 | 1 | 1 | 1 |
| 22 | 1 | 0 | 1 | 0 | 2 | 1 | 1 |
| 23 | 1 | 0 | 1 | 0 | 1 | 0 | 0 |
| 24 | 1 | 0 | 1 | 1 | 0 | 0 | 0 |
| 25 | 1 | 0 | 1 | 0 | 1 | 0 | 1 |
| 26 | 1 | 0 | 1 | 1 | 0 | 0 | 1 |

| | LacdiNAc | Sia-LacdiNAc | | | | | |
|---|---|---|---|---|---|---|---|
| Compound | on complex | on complex | Oligomannosidic | Hybrid | | Mass | [M + H]⁺ |
| ID | diantennary | diantennary | glycans | glycans | Sulfation | [Daltons] | [m/z] |
| 1 | 0 | 0 | 1 | 0 | 0 | 1476.57 | 1477.58 |
| 2 | 0 | 0 | 0 | 0 | 0 | 1808.69 | 1809.70 |
| 3 | 0 | 0 | 0 | 0 | 0 | 1954.75 | 1955.76 |
| 4 | 0 | 0 | 0 | 0 | 0 | 1954.75 | 1955.76 |
| 5 | 0 | 0 | 0 | 0 | 0 | 1995.78 | 1996.78 |
| 6 | 0 | 0 | 0 | 1 | 0 | 2132.80 | 2133.81 |
| 7 | 0 | 0 | 0 | 0 | 0 | 2157.83 | 2158.84 |
| 8 | 0 | 0 | 0 | 0 | 0 | 2173.83 | 2174.83 |
| 9 | 0 | 0 | 0 | 0 | 0 | 2319.88 | 2320.89 |
| 10 | 0 | 0 | 0 | 0 | 0 | 2319.88 | 2320.89 |
| 11 | 1 | 0 | 0 | 0 | 0 | 2360.91 | 2361.92 |
| 12 | 0 | 0 | 0 | 0 | 0 | 2464.92 | 2465.93 |
| 13 | 0 | 0 | 0 | 0 | 0 | 2464.92 | 2465.93 |
| 14 | 0 | 0 | 0 | 0 | 0 | 2464.92 | 2465.93 |
| 15 | 1 | 1 | 0 | 0 | 0 | 2505.95 | 2506.95 |
| 16 | 1 | 1 | 0 | 0 | 0 | 2505.95 | 2506.95 |
| 17 | 0 | 0 | 0 | 0 | 0 | 2610.98 | 2611.99 |
| 18 | 0 | 0 | 0 | 0 | 0 | 2610.98 | 2611.99 |
| 19 | 0 | 0 | 0 | 0 | 0 | 2610.98 | 2611.99 |
| 20 | 1 | 1 | 0 | 0 | 0 | 2652.01 | 2653.01 |
| 21 | 1 | 1 | 0 | 0 | 0 | 2652.01 | 2653.01 |
| 22 | 2 | 2 | 0 | 0 | 0 | 2693.03 | 2694.04 |
| 23 | 1 | 0 | 0 | 0 | 1 | 2294.81 | 2295.82 |
| 24 | 1 | 0 | 0 | 0 | 1 | 2294.81 | 2295.82 |
| 25 | 1 | 0 | 0 | 0 | 1 | 2440.87 | 2441.87 |
| 26 | 1 | 0 | 0 | 0 | 1 | 2440.87 | 2441.87 |

The present invention in particular also provides for the following embodiments:
1. A method to aid in determining whether or not a subject has an aggressive form of prostate cancer, comprising:
   (a) determining the level of at least one mono-antennary PSA glycoform and the level of at least one di-antennary PSA glycoform in a biofluid sample from the subject;
   (b) calculating an aggressiveness score for the subject based on the levels determined in (a);
   (c) comparing the subject's aggressiveness score in (b) to a reference value for said aggressiveness score; and
   (d) using the comparison in (c) to aid in determining whether the subject has an aggressive form of prostate cancer or not.
2. A method for determining whether or not a subject has an aggressive form of prostate cancer, comprising:
   (i) isolating PSA protein from other proteins in a biofluid sample from the subject;
   (ii) treating the isolated PSA protein with a protease;
   (iii) determining the level of each of two or more N₆₉ glycopeptide forms in the sample, wherein at least one of the N₆₉ glycopeptide forms is a mono-antennary glycoform and at least one of the N₆₉ glycopeptide forms is a di-antennary glycoform;
   (iv) determining an aggressiveness score for the subject using the levels of each N₆₉ glycopeptide forms in (iii);
   (v) comparing the aggressiveness score score in (iv) to a reference value for said aggressiveness score; and
   (vi) based on the comparison in step (v) determining whether the subject has an aggressive form of PCa or not.
3. The method according to embodiment 2, wherein the protease is selected from trypsin and Arg-C (clostripain).
4. The method according to embodiment 1, 2 or 3, wherein if the subject is not determined to have an aggressive form of PCa they are determined to have indolent prostatic disease.
5. The method according to any one of embodiments 1-4, wherein the at least on mono-antennary PSA glycoform is selected from the group consisting of: H4N3S_{2,6}1; H4N3F1S_{2,6}1; H4N3F1 S_{2,3}1; and H3N4F1S_{2,6}1.
6. The method according to any one of embodiments 1-5, wherein the at least one di-antennary PSA glycoform is selected from the group consisting of: H4N4F1S_{2,6}1; H5N4F1S_{2,6}1; H5N4F1S_{2,3}1; H4N5F1S_{2,6}1; H5N4S_{2,3}1 S_{2,6}1; H4N5S_{2,3}1 S_{2,6}1; H4N5F1S_{2,3}1S_{2,6}1; H4N5S_{2,3}1(SO₃)1; and H4N5F1S_{2,6}1 (SO₃)1.
7. The method according to any one of the preceding embodiments, wherein the level of 3, 4, 5, or more PSA glycoforms are determined.
8. The method according to any one of the preceding embodiments, wherein the level of each PSA glycoform determined is normalised.
9. The method according to any one of the preceding embodiments, wherein the aggressiveness score comprises or consists of a ratio of the level of the at least one di-antennary PSA glycoform from the subject sample over the at least one mono-antennary PSA glycoform or a vice versa ratio.
10. The method according to any one of the preceding embodiments, wherein the aggressiveness score is a binary score comprising a first value corresponding to the level of the at least one mono-antennary PSA glycoform and a second value corresponding to the level of the at least one di-antennary PSA glycoform, wherein the reference value for the aggressiveness score comprises a reference value for the first value and a reference value for the second value, and wherein if the level of the at least one mono-antennary PSA glycoform is decreased compared to reference value for the first value and the level of the at least one di-antennary PSA glycoform is increased compared to reference value for the second value, the subject is determined to have an aggressive form of PCa.
11. The method according to any one of the preceding embodiments, wherein the PSA glycoform(s) used or determined in the methods of the invention comprise PSA glycoform(s) selected from a group consisting of:
   (i) H4N3F1S_{2,6}1 and H4N4F1S_{2,6}1;
   (ii) H4N3F1S_{2,6}1 and H5N4F1S_{2,6}1;
   (iii) H4N3S_{2,6}1 and H4N5S_{2,3}1(SO₃)1;
   (iv) H4N3S_{2,6}1 and H5N4F1S_{2,3}1S_{2,6}1;
   (v) H4N3S_{2,6}1 and H4N4F1S_{2,6}1;
   (vi) H4N3F1S_{2,6}1 and H5N4F1S_{2,3}1S_{2,6}1;
   (vii) H4N3S_{2,6}1 and H4N5F1S_{2,3}1S_{2,6}1;
   (viii) H4N3S_{2,6}1 and H4N5F1S_{2,6}1;
   (ix) H3N4F1S_{2,6}1 and H4N5F1S_{2,3}1S_{2,6}1.
12. The method according to any one of embodiments 1-10, wherein the PSA glycoforms used or determined in the methods of the invention comprise PSA glycoforms selected from a group consisting of:
   a. H4N4F1S_{2,6}1 and a combination of mono-antennary glycoforms;
   b. H4N3S_{2,6}1 and a combination of mono-antennary glycoforms;
   c. H4N3S_{2,6}1 and a combination of α2.3.sialylation glycoforms;
   d. H4N3S_{2,6}1 and a combination of α2.6.sialylation glycoforms;
   e. H5N4F1S_{2,3}1S_{2,6}1 and a combination of mono-antennary glycoforms;
   f. H5N4F1S_{2,3}1 and a combination of mono-antennary glycoforms;
   g. a combination of α2.3.sialylation glycoforms and a combination of mono-antennary glycoforms;
   h. H4N5F1S_{2,3}1S_{2,6}1 and a combination of mono-antennary glycoforms; and
   i. H4N3F1S_{2,3}1 and a combination of mono-antennary glycoforms.
13. A method to aid in determining whether or not a subject has an aggressive form of prostate cancer, comprising:
   (a) determining the level of a PSA glycoform selected from the group consisting of: H4N5F1S_{2,3}1S_{2,6}1; H4N3S_{2,6}1; and H5N4F1S_{2,6}1 in a biofluid sample from the subject,
   (b) comparing the value for the level of the PSA glycoform in (a) to a reference value for said PSA glycoform; or comparing an aggressiveness score taking into account the level said PSA glycoform in (a) to a reference value for said aggressiveness score; and
   (c) using the comparison in (b) to aid in determining whether the subject has an aggressive form of prostate cancer or not.
14. The method according to any one of the preceding embodiments, wherein the subject is a mammal, such as a male human.
15. The method according to any one of the preceding embodiments, wherein the biofluid sample is selected from: blood, serum, plasma, seminal fluid, prostatic fluid and urine.
16. The method according to embodiment 15, wherein the sample is a urine sample, optionally a digital rectal examination (DRE) urine sample.
17. The method according to any one of the preceding embodiments, wherein the subject is preselected based having prostate-gland enlargement.
18. The method according to any one of the preceding embodiments, wherein the subject is preselected based on the level of total serum PSA.
19. The method according to embodiment 18, wherein the level of total serum PSA in the subject is between about 2-10 ng/ml.
20. The method according to any one of the preceding embodiments, wherein the PSA protein in the subject's biofluid sample is isolated from the sample.
21. The method according to embodiment 20, wherein the PSA is isolated from the sample by use of a capture binding partner, such as an anti-PSA antibody.
22. The method according to embodiment 20 or 21, wherein the isolated PSA protein is treated with a protease, such as trypsin or Arg-C (clostripain), to produce N₆₉ comprising glycopeptides.
23. The method according to any one of embodiments 20 to 22, wherein the isolated PSA or N₆₉ comprising glycopeptides are treated with a glycosidase to release the glycoforms from the PSA protein or N₆₉K di-peptide.
24. The method according to any one of the preceding embodiments, wherein the level of each measured PSA glycoform is determined by mass spectrometry, such as CE-ESI-MS or LC-ESI-MS(/MS), or MALDI-TOF-MS
25. The method according to any of the preceding embodiments, wherein the determining is used to provide a treatment recommendation for the patient and/or to decide whether a prostate biopsy is needed.
26. The method according to any one of embodiments 1-12 and 14-25, wherein the reference value for the aggressiveness score has been determined from samples of at least one reference population comprising aggressive PCa subjects and subjects that do not have aggressive PCa, such as patients with prostatitis, BPH or indolent PCa, optionally wherein said disease status is known.
27. A method according to any one of claims 13-25, wherein a reference value for said PSA glycoform has been determined from samples of at least one reference population comprising aggressive PCa subjects and subjects that do not have aggressive PCa, such as patients with prostatitis, BPH or indolent PCa, optionally wherein said disease status is known.
28. A kit for use in a method according to any one of embodiments 1 to 27, comprising a scoring system with PSA glycoform reference values or ratios indicative of aggressive PCa, and optionally instructions for use.
29. The kit according to embodiment 28, also comprising a PSA binding partner, such as a monoclonal antibody, optionally wherein the PSA binding partner is immobilised onto a solid surface, such as a bead.
30. Use of a kit in a method according to any one of embodiments 1 to 27, comprising a scoring system with PSA glycoform reference values or ratios indicative of aggressive PCa, and optionally instructions for use.
31. The use according to embodiment 30, also comprising a PSA binding partner, such as a monoclonal antibody, optionally wherein the PSA binding partner is immobilised onto a solid surface, such as a bead.
32. A computer-implemented method to aid in determining whether a subject has an aggressive form of prostate cancer or not, comprising the steps of:
   (a) receiving a value for the level of a first mono-antennary PSA glycoform in a biofluid sample of the subject;
   (b) receiving a value for the level of a first di-antennary PSA glycoform in the biofluid sample of the subject;
   (c) calculating an aggressiveness score for the subject based on the levels received in (a) and (b);
   (d) comparing the subject's aggressiveness score in (c) to a reference value for said aggressiveness score; and
   (e) using the comparison in (d) to aid in determining whether the subject has an aggressive form of prostate cancer or not.
33. A computer-implemented method to aid in determining whether a subject has an aggressive form of prostate cancer or not, comprising the steps of:
   (a) receiving a value for the level of a PSA glycoform selected from the group consisting of H4N5F1 S_{2,3}1S_{2,6}1; H4N3S_{2,6}1; and H5N4F1S_{2,6}1 in a biofluid sample of the subject;
   (b) comparing the value for the level of the PSA glycoform received in (a) to a reference value for said PSA glycoform; or an aggressiveness score taking into account the level of the PSA glycoform received in (a) to a reference value for said aggressiveness score; and
   (c) using the comparison in (b) to aid in determining whether or not the subject has an aggressive form of prostate cancer.

Further aspects and embodiments will be apparent to those skilled in the art. All documents mentioned in this text are incorporated herein by reference. Aspects and embodiments of the present invention will now be discussed with reference to the following Examples and accompanying Figures.

### FIGURES

FIG. 1 is a schematic overview of the PSA glycomics assay. Step 1 includes the sample collection of the urine samples. Step 2 isolates the PSA from the urine. Step 3 prepares the sample for analysis by reducing and alkylating the PSA followed by an in-solution digestion (e.g. using trypsin). After sample preparation the sample can be by analysed by capillary electrophoresis (step 4.A) followed by mass spectrometry for detection (step 4.B). After the analysis the data is collected and further processed (step 5).

FIG. 2. AUC ROC of urinary Comp. 21 and serum fPSA % for differentiation of the aggressive (Gleason ≥7) vs indolent PCa and BPH.

FIG. 3. AUC ROC of the best urinary 2-compound combination (Comp. 3+7) and serum fPSA % for differentiation of the aggressive vs indolent PCa and BPH.

FIG. 4. AUC ROC of the best urinary 3-compound combination (Comp. 4+7+mono-antennary glycans) and serum fPSA % for differentiation of the aggressive vs indolent PCa and BPH.

FIG. 5. AUC ROC of the best urinary 4-compound combination (Comp. 2+9+13+22) and serum fPSA % for differentiation of the aggressive vs indolent PCa and BPH.

FIG. 6. Major glycoforms with same group traits (up- or down-regulated). Structural glycosylation traits (e.g. sialylation, fucosylation, and branching type) are annotated in the left column. Structures description is given in the right column under “Glycan structure”. “1” denotes presence of specific trait for given glycan, “0” denotes absence of specific trait for given glycoform.

FIG. 7. AUC ROC of the best ratio between up- and down-regulated urinary PSA glycostructures and serum fPSA % for differentiation of the aggressive vs indolent PCa and BPH.

FIGS. 8-18 depict exemplary structures of various PSA glycoform (compounds) of the invention attached to peptide (PEP). The right representation uses the SNFG (Symbol Nomenclature for Glycans) system (Ajit Varki et al., Symbol Nomenclature for Graphical Representations of Glycans, Glycobiology, Volume 25, Issue 12, December 2015, Pages 1323-1324, https://doi.org/10.1093/glycob/cwv091 and Sriram Neelamegham et a. al, The SNFG Discussion Group, Updates to the Symbol Nomenclature for Glycans guidelines, Glycobiology, Volume 29, Issue 9, September 2019, Pages 620-624, https://doi.org/10.1093/glycob/cwz045). The linkage type of sialic acid (2,3 or 2,6) is indicated in Table 1A above.

FIG. 8—depicts an exemplary compound 2 structure.

FIG. 9—depicts an exemplary compound 3 structure.

FIG. 10—depicts an exemplary compound 4 structure.

FIG. 11—depicts an exemplary compound 5 structure.

FIG. 12—depicts an exemplary compound 7 structure.

FIG. 13—depicts an exemplary compound 9 structure.

FIG. 14—depicts an exemplary compound 13 structure.

FIG. 15—depicts an exemplary compound 18 structure.

FIG. 16—depicts an exemplary compound 21 structure.

FIG. 17—an exemplary compound 22 structure.

FIG. 18—depicts an exemplary compound 24 structure.

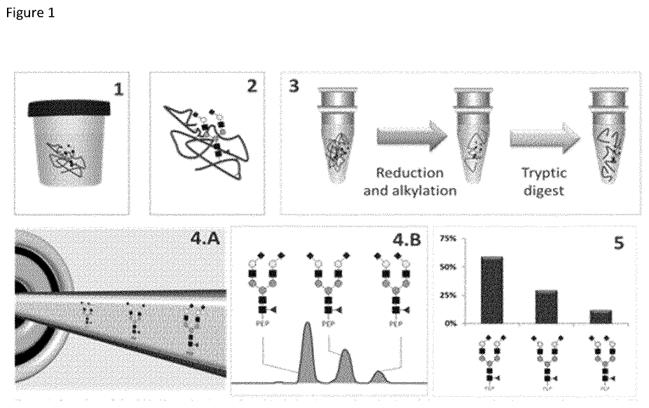

### EXAMPLES

### Example 1

### 1.1 Abbreviations

HAc Acetic acid
BGE Background electrolyte
CE-ESI-MS Capillary electrophoresis—electrospray ionization—mass spectrometry using a sheathless porous sprayer
PSA Prostate-Specific Antigen
BPH Benign Prostatic Hyperplasia
LE Leading Electrolyte
AmAc Ammonium acetate
PCa Prostate cancer
RT Room temperature
ABC Ammonium bicarbonate
FA Formic acid
PGA PSA Glycomics Assay
FUP Female urine pool
DTT Dithiothreitol
IAA lodoacetamide
Serum fPSA %% of free-PSA to total PSA in serum

### 1.2 Materials

### 1.2.1 Clinical Samples

A total of 110 digital rectal examination (DRE) urine samples of 106 patients with elevated and “grey-zone” PSA concentrations in serum (total PSA between 2-10 ng/ml) were analysed with the optimized PSA Glycomics Assay (PGA). The panel included four replicates for the technical robustness evaluation.

DRE urine samples obtained from prospective multicentric studies were analysed. The sample panel was composed of urine from 33 benign controls (benign prostatic hyperplasia; BPH) and 73 PCa patients. Prostate cancer (PCa) samples are further divided into indolent PCa (Gleason score ≤6, n=29) and aggressive PCa (Gleason ≥7, n=44). The majority of PCa patients had total PSA serum values between 2-10 ng/ml, thus representing a grey zone population (see Table 2 and 3 for demographic and clinical information of sample panel). One mL DRE urine was used for PGA approach.

All samples were diluted prior to the capture procedure to 100 ng/mL PSA unless the concentration was below this threshold (based on total PSA Elecsys assay—Roche Diagnostics GmbH, Mannheim, Germany). Dilution was performed with a female urine pool (FUP) that was created by pooling urine samples from healthy female volunteers.

### 1.2.2 Positive Control

PSA-protein standard derived from human seminal fluid (obtained from Lee BioSolutions—St. Louis, MO) was used as a positive control. A positive control sample was created by adding 100 ng of the PSA-protein standard to FUP.

### 1.2.3 Negative Control

As a negative control, FUP was used from the urine samples obtained from healthy female volunteers.

### 1.2.4 System Suitability Sample

A tryptic digest of the PSA-protein standard was used as a system suitability sample to test if the analytical performance was as expected prior to analysing the DRE urine samples. For this purpose, several aliquots were prepared containing 50 ng of PSA-protein standard and the internal peptide standard (see Table 4), followed by reduction, alkylation and trypsin digestion of the protein. After digestion the samples were pooled and redistributed over the same amount of aliquots (see Kammeijer et al. Anal. Chem. 90, 4414-4421, 2018, for protocol).

TABLE 2
Patient demographics, stratified by PCa vs BPH

| | | |
|---|---|---|
| BPH(N = 33) | PCa(N = 73) | Total (N = 106) |

Age

| | | | |
|---|---|---|---|
| Mean | 67 | 66 | 66 |
| SD | 6.9 | 7.1 | 7 |
| Median | 68 | 67 | 68 |
| P25-P75 | 64-71 | 61-71 | 62-71 |
| Min-Max | 48-76 | 42-78 | 42-78 |
| Missing, % | 0(0%) | 0(0%) | 0(0%) |
| N | 33 | 73 | 106 |

DRE Positive

| | | | |
|---|---|---|---|
| no | 25(83%) | 69(95%) | 94(91%) |
| yes | 5(17%) | 4(5.5%) | 9(8.7%) |

Prostate Volume [mL]

| | | | |
|---|---|---|---|
| Mean | 49 | 38 | 41 |
| SD | 30 | 18 | 23 |
| Median | 40 | 30 | 35 |
| P25-P75 | 33-50 | 27-40 | 28-45 |
| Min-Max | 20-170 | 15-110 | 15-170 |
| Missing, % | 6(18%) | 8(11%) | 14(13%) |
| N | 27 | 65 | 92 |

PSA in Urine [ng/mL]

| | | | |
|---|---|---|---|
| Mean | 2714 | 1905 | 2157 |
| SD | 2389 | 2205 | 2284 |
| Median | 2216 | 995 | 1199 |
| P25-P75 | 402-5604 | 209-2166 | 273-3548 |
| Min-Max | 5.1-6025 | 19-7166 | 5.1-7166 |
| Missing, % | 0(0%) | 0(0%) | 0(0%) |
| N | 33 | 73 | 106 |

Gleason score

| | | | |
|---|---|---|---|
| 6 | 0(NA %) | 29(40%) | 29(40%) |
| 7 | 0(NA %) | 12(16%) | 12(16%) |
| 8 | 0(NA %) | 12(16%) | 12(16%) |
| 9 | 0(NA %) | 18(25%) | 18(25%) |
| 10 | 0(NA %) | 2(2.7%) | 2(2.7%) |

TABLE 3
Patient demographics, stratified by PCa aggressiveness.

| | | | |
|---|---|---|---|
| BPH(N = 33) | indolent(N = 29) | aggressive(N = 44) | Total (N = 106) |

Age

| | | | | |
|---|---|---|---|---|
| Mean | 67 | 64 | 67 | 66 |
| SD | 6.9 | 7.6 | 6.6 | 7 |
| Median | 68 | 64 | 68 | 68 |
| P25-P75 | 64-71 | 57-71 | 64-71 | 62-71 |
| Min-Max | 48-76 | 51-78 | 42-77 | 42-78 |
| Missing, % | 0(0%) | 0(0%) | 0(0%) | 0(0%) |
| N | 33 | 29 | 44 | 106 |

DRE Positive

| | | | | |
|---|---|---|---|---|
| no | 25(83%) | 29(100%) | 40(91%) | 94(91%) |
| yes | 5(17%) | 0(0%) | 4(9.1%) | 9(8.7%) |

Prostate Volume [mL]

| | | | | |
|---|---|---|---|---|
| Mean | 49 | 40 | 37 | 41 |
| SD | 30 | 17 | 19 | 23 |
| Median | 40 | 37 | 30 | 35 |
| P25-P75 | 33-50 | 29-43 | 26-36 | 28-45 |
| Min-Max | 20-170 | 20-80 | 15-110 | 15-170 |
| Missing, % | 6(18%) | 3(10%) | 5(11%) | 14(13%) |
| N | 27 | 26 | 39 | 92 |

PSA in Urine [ng/mL]

| | | | | |
|---|---|---|---|---|
| Mean | 2714 | 2109 | 1770 | 2167 |
| SD | 2389 | 2130 | 2266 | 2284 |
| Median | 2216 | 1462 | 758 | 1199 |
| P26-P75 | 402-5604 | 401-2886 | 121-2129 | 273-3548 |
| Min-Max | 5.3-6025 | 30-6025 | 19-7166 | 5.1-7166 |
| Missing, % | 0(0%) | 0(0%) | 0(0%) | 0(0%) |
| N | 33 | 29 | 44 | 106 |

### 1.3 Methods

The 110 patient samples were randomly divided over six batches. One positive and one negative control was included in each batch.

### 1.3.1 Anti-PSA Beads IgG-M36

Biotinylated monoclonal IgG clone M36 anti-PSA antibody was obtained from Roche Diagnostics GmbH, Mannheim, Germany. Six mg (3 mg/mL) of antibody solution was added to 600 μL of washed streptavidin beads (ThermoFischer) and incubated overnight at 4° C. with continuous mixing. The next day the beads were washed with 1×PBS to remove unbound antibodies. Non-specific bound antibodies were removed by incubating the beads for 5 minutes in 100 mM formic acid (FA). The pH was readjusted to 7 and the beads were stored as a 50% slurry in 1×PBS containing 0.02% sodium azide (NaN₃) as a preservative at 4° C. Such beads are ready for use without prewashing.

### 1.3.2 PSA Capturing

Samples were thawed, mixed by hand and spun for 1 min at 500 g to pellet and remove precipitate. Supernatant was collected and used for further analysis and the samples were placed on ice. Dilutions were made based upon urinary PSA concentrations to a concentration of 100 ng/mL and added up to 1 mL with FUP. If the concentration was below 100 ng/mL the full sample was used.

If sufficient sample was left over, an additional 20 ng of each urine sample was taken to generate a pooled sample. For all experimental steps samples and reagents were kept on ice to keep the PSA as stable as possible before the start of the capturing procedure.

In total, 250 μL 5×PBS and 2 μL of 50% antibody-beads suspension (IgG-M36) were added to the sample, capturing was performed overnight at 4° C. Next, the beads were washed 1x with PBS (pH 7-8) and 2x with ammonium bicarbonate (ABC) (pH 8). Prior to elution an internal standard (Table 4) was added to the sample well. The PSA was eluted with 100 mM FA (pH 2-3). After elution the samples were dried.

TABLE 4
Synthetic peptide (internal standard) added to
sample prior to elution and their resemblance to
the endogenous peptide.

| Peptide | Peptide sequence | SEQ ID NO: |
|---|---|---|
| Endogenous peptide | LSEPAELTDAVK | 1 |
| Internal peptide | LSEPAELTEAVK | 2 |
| standard | | |

### 1.3.3 Digestion

The samples were digested as described by Kammeijer et al. (supra). Briefly, the dried eluate of the captured PSA glycoprotein samples were reconstituted in 25 mM ammonium bicarbonate (ABC) buffer. Reduction was carried out for 30 min at 60° C. with dithiothreitol (DTT) in a final concentration of 2 mM. The sample was cooled down at room temperature (RT) and sulfide alkylation was performed using iodoacetamide (IAA) for 30 min in the dark at RT (final concentration of 6 mM). To ensure that the alkylation process was stopped, additional DTT was added (final concentration 6 mM). For overnight digestion at 37° C., 0.15 ug porcine trypsin was added.

### 1.3.4 Equipment

All capillary electrophoresis (CE) experiments were carried out on a CESI 8000 system (SCIEX Separations, Framingham, MA), either 91-cm long bare fused capillaries, dynamic coated neutral capillaries or static coated neutral capillaries (all with an internal diameter of 30 μm and outer diameter of 150 μm). Prior to analysis, the capillary was rinsed thoroughly with 0.1 M NaOH (2.5 min), 0.1 M HCl (2.5 min), water (4 min) and background electrolyte (BGE) which consisted of 10% acetic acid (HAc) (v/v, pH 2.3).

For the analysis an on-line pre-concentration step was used (e.g. transient isotachophoresis (t-ITP)). Prior to injection, ammonium acetate (AmAc) was added to the sample (400 mM). The AmAC acted as leading electrolyte. For all experiments, samples were injected with 25 psi for 24 sec (corresponding to approximately 13.5% of the capillary volume), followed by a BGE post plug by applying 0.5 psi for 25 sec (corresponding to approximately 0.3% of the capillary volume). Separation was carried out by applying a voltage of 20 kV.

The CE system was coupled to a UHR-QqTOF maXis Impact HD MS (Bruker Daltonics) via a sheathless CE-ESI-MS interface from SCIEX. A capillary voltage of between −1000 and −1300 V was applied to ensure a stable electrospray. All experiments were performed in positive ionization mode. The drying gas (nitrogen) flow rate and temperature were set at 1.5 L/min and 150° C., respectively. MS data was acquired between m/z 200-2200 with a spectral acquisition rate of 1 Hz.

### 1.3.5 Data Analysis

CE-ESI-MS data was analysed with DataAnalysis (Bruker Daltonics). Prior to data analysis, all MS spectra were calibrated using sodium adducts detected at the beginning of the electropherogram. The data was manually screened for an endogenous peptide (Table 4) and a spiked internal standard I (synthetic peptide in which one amino acid was substituted from the endogenous peptide, see Table 4). Extracted ion electropherograms (EIEs, smoothed with a Gaussian fit) were acquired with the first three isotopes of the doubly and triply charged analytes using a width of m/z±0.05 unit.

### 1.4 Results

### 1.4.1 System Suitability

The peak areas (summed CE-MS intensities in MS1) observed for the endogenous peptide and spiked internal standard I of the system suitability standards revealed that the system was operating well throughout the full measurement of all samples (data not shown).

### 1.4.2 Clinical Evaluation of PSA Glycans

Measurement of 110 clinical samples resulted in identification of 26 N-glycopeptides (areas represented as relative peak area [%], normalized to all identified glycoforms summed up to 100%).

Areas of individual glycoforms were grouped based on same glycosylation traits, e.g. fucosylation, mono-sialylation, di-sialylation, α2,3-sialylation, α2,6-sialylation or sulfation (as indicated in Table 1 B and identified as “summed” in Table 5), were used in univariate and multivariate biostatistical analysis to identify a structure (biomarker) or composition of several structures (biomarker panel) providing best differentiation of aggressive PCa from indolent PCa and BPH. See Table 4 for list of individual and combined glycan biomarkers.

Table 1 B indicates to which category/glycosylation feature a particular glycoform possesses (e.g. whether it is mono-antennary, di-antennary, is fucosylated, has α2,3-sialylation etc.). Where the Comp ID is marked as Summed (Yes) in Table 4 above, it means that each member of that class (as in Table 1B) was measured and summed together (e.g. Comp.Sulfation=compounds 23, 24, 25 and 26).

Clinical performance of the urinary glycoforms or glycosylation category was compared to the efficacy of serum free PSA % (Elecsys serum free PSA %=free PSA/total PSA) in the same cohort.

Biostatistical Analysis were Performed as Follows:

Univariate analysis were based on: (1) p-values from Wilcoxon tests, (2) false discovery rate (FDR) of 30% and (3) biomarker performance illustrated by Area under Receiver Operating Characteristic (AUC ROC) curve.

Multivariate analysis included (1) ROC analysis for all linear combinations in 2, 3, and 4 marker Panels (1× cross validated) and (2) selection frequency of combinations from 2× cross validation (inner loop: feature selection, outer loop: performance estimate, 100 splits in training and test set in outer loop).

Furthermore, as deduced from multivariate analysis, best combinations are mostly direction +/−(increased compound and decreased compound) or −/+(decreased compound and increased compound). Therefore, the ratios between the best up-regulated (Comp. 2, 3, 4, 5) and down-regulated compound groups (Comp. 7, 9, 18, 24; FIG. 6) were calculated and their clinical performance estimated in the study population.

TABLE 5
Identified individual glycoforms and combined compositions
indicated as “no” and “yes” in column Summed,
respectively. Combined compositions = all glycoforms
having said feature according to Table 1A and 1B; combination
of levels is achieved by building a simple mathematical sum.

| CompID | Glycan_structure | Summed |
|---|---|---|
| Comp. 1 | HSN2 | No |
| Comp. 2 | H4N3S2,61 | No |
| Comp. 3 | H4N3F1S2,61 | No |
| Comp. 4 | H4N3F1S2,31 | No |
| Comp. 5 | H3N4F1S2,61 | No |
| Comp. 6 | H6N3S2,61 | No |
| Comp. 7 | H4N4F1S2,61 | No |
| Comp. 8 | H5N4S2,61 | No |
| Comp. 9 | H5N4F1S2,61 | No |
| Comp. 10 | H5N4F1S2,31 | No |
| Comp. 11 | H4N5F1S2,61 | No |
| Comp. 12 | H5N4S2,62 | No |
| Comp. 13 | H5N4S2,31S2,61 | No |
| Comp. 14 | H5N4S2,32 | No |
| Comp. 15 | H4N5S2,62 | No |
| Comp. 16 | H4N5S2,31S2,61 | No |
| Comp. 17 | H5N4F1S2,62 | No |
| Comp. 18 | H5N4F1S2,31S2,61 | No |
| Comp. 19 | H5N4F1S2,32 | No |
| Comp. 20 | H4N5F1S2,62 | No |
| Comp. 21 | H4N5F1S2,31S2,61 | No |
| Comp. 22 | H3N6F1S2,62 | No |
| Comp. 23 | H4N5S2,61(SO3)1 | No |
| Comp. 24 | H4N5S2,31(SO3)1 | No |
| Comp. 25 | H4N5F1S2,61(SO3)1 | No |
| Comp. 26 | H4N5F1S2,31(SO3)1 | No |
| Comp. a2,3-sialylation | a2,3-sialylation | Yes |
| Comp. a2,6-sialylation | a2,6-sialylation | Yes |
| Comp. Di-sialylation | Di-sialylation | Yes |
| Comp. Fucosylation | Fucosylation | Yes |
| Comp. LacdiNAc | LacdiNAc | Yes |
| Comp. Sia-LacdiNAc | Sia-LacdiNAc | Yes |
| Comp. High mannose glycans | High mannose glycans | Yes |
| Comp. Hybrid glycans | Hybrid glycans | Yes |
| Comp. Mono-anntenary | Mono-anntenary | Yes |
| Comp. Sulfation | Sulfation | Yes |

### 1.4.3 Results Univariate Analysis

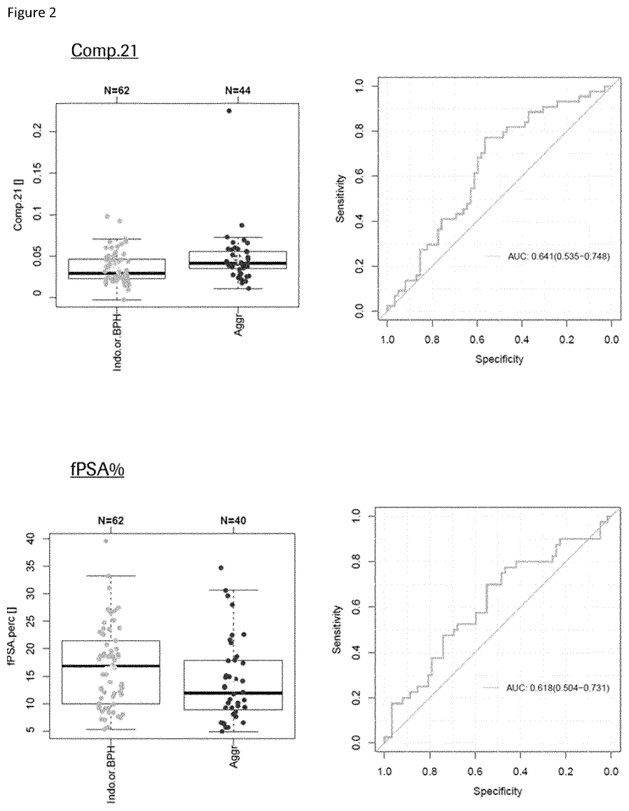

Univariate analysis of all 26 different glycoforms and 10 combined glycosylation features revealed good clinical performance (discrimination ability) of the PSA glycoforms, with at least three glycoforms. The AUG ROG for differentiation of aggressive vs indolent PGa and BPH were higher compared to serum fPSA %. Particularly compound (Gomp.) 21 with AUG 64.1, Gomp. 2 with AUG 63.0 and Gomp. 9 with AUG 62.0 performed better as serum fPSA % with AUG 61.8. See Table 6 for detailed compound structures and FIG. 2 for comparison of AUG ROG for Gomp. 21 to serum free PSA %.

TABLE 6
Univariate performance of all individual glycoforms and combined
PSA glycoform categories ranked by their AUC value for differentiation
of aggressive from indolent PCa and BPH. Direction indicates
increased (+) or decreased (−) intensity of given glycoform
or category in Gleason ≥7 PCa. fPSA %: serum free PSA %.

| CompID | Glycan_structure | corr.p.value | AUC |
|---|---|---|---|
| Comp. 21 | H4N5F1S2,31S2,61 | 2.75e−1 | 64.1 |
| Comp. 2 | H4N3S2,61 | 2.75e−1 | 63 |
| Comp. 9 | H5N4F1S2,61 | 2.75e−1 | 62 |
| fPSA.perc | | 2.75e−1 | 61.8 |
| Comp. 18 | H5N4F1S2,31S2,61 | 2.75e−1 | 61.6 |
| Comp. 14 | H5N4S2,32 | 2.75e−1 | 60.7 |
| Comp. 11 | H4N5F1S2,61 | 2.75e−1 | 60.5 |
| Comp. 20 | H4N5F1S2,62 | 2.75e−1 | 60.3 |
| Comp. 19 | H5N4F1S2,32 | 2.75e−1 | 60.1 |
| Comp. 12 | H5N4S2,62 | 2.75e−1 | 59.9 |
| Comp. 22 | H3N6F1S2,62 | 2.75e−1 | 59.9 |
| Comp. Sulfation | Sulfation | 2.75e−1 | 59.2 |
| Comp. 24 | H4N5S2,31(SO3)1 | 2.75e−1 | 59.1 |
| Comp. 3 | H4N3F1S2,61 | 2.75e−1 | 59.1 |
| Comp. 7 | H4N4F1S2,61 | 2.75e−1 | 59.1 |
| Comp. a2,3-sialylation | a2,3-sialylation | 2.76e−1 | 58.9 |
| Comp. Di-sialylation | Di-sialylation | 2.76e−1 | 58.7 |
| Comp. 26 | H4N5F1S2,31(SO3)1 | 2.76e−1 | 58.5 |
| Comp. a2,6-sialylation | a2,6-sialylation | 2.76e−1 | 58.4 |
| Comp. Mono-anntenary | Mono-anntenary | 2.76e−1 | 58.4 |
| Comp. Fucosylation | Fucosylation | 2.82e−1 | 58.3 |
| Comp. 10 | H5N4F1S2,31 | 2.89e−1 | 58.1 |
| Comp. LacdiNAc | LacdiNAc | 2.89e−1 | 58.1 |
| Comp. Sia-LacdiNAc | Sia-LacdiNAc | 2.92e−1 | 58 |

### 1.4.4 Results Multivariate Analysis

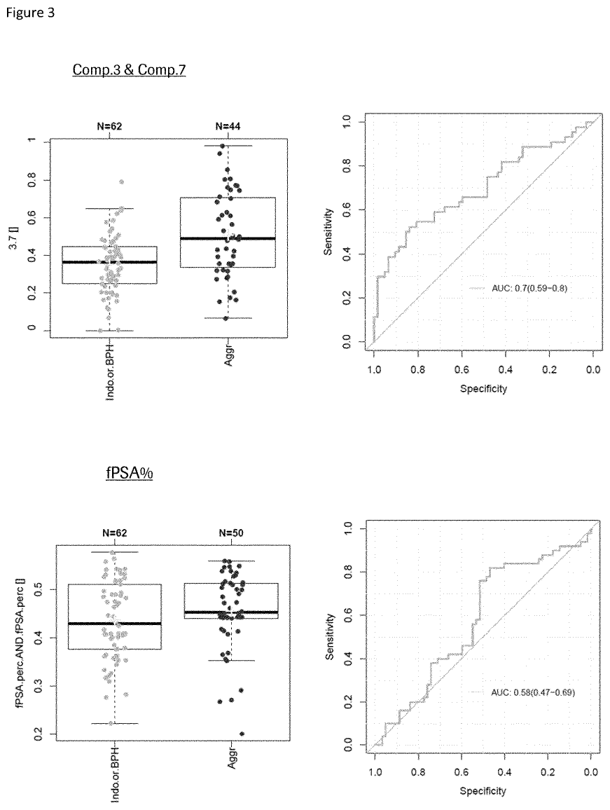

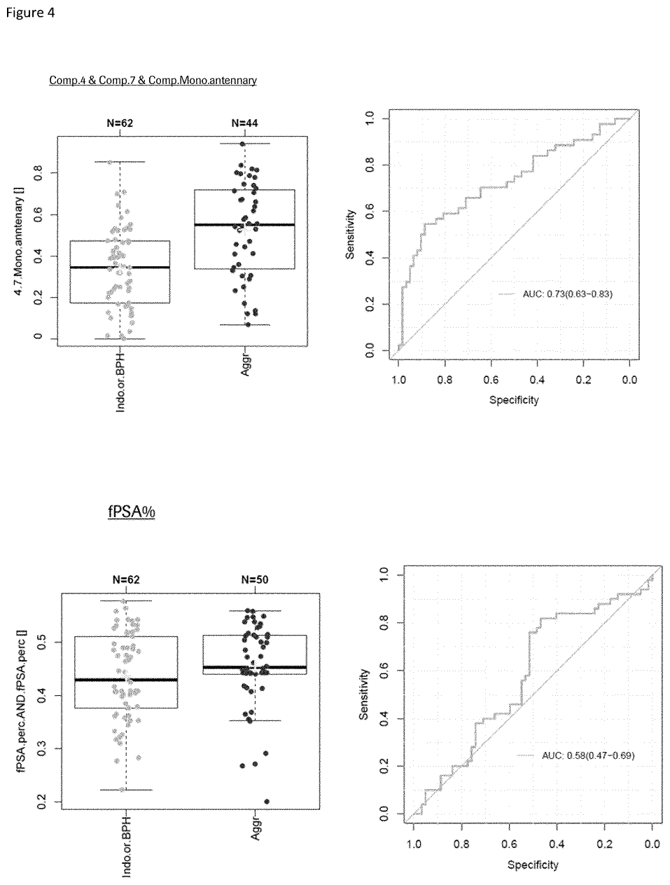

In multivariate analyses combining 2, 3 or 4 compounds or compound groups all glycoform combinations performed better in differentiation of aggressive vs indolent PCa and BPH compared to serum fPSA % (Tables 7-9 and FIGS. 3-5). The addition of further compounds to the combination panel steadily increased panel performance from AUC 69.8 for two structures (Comp. 3+7); AUC 73.0 for three structures (Comp. 4+7+mono-antennary); and AUC 75.1 for combination of the four structures (Comp. 2+9+13+22).

TABLE 7
Multivariate performance of bivariate 2-compound combinations ranked according their AUC value
for differentiation of aggressive from indolent PCa and BPH. fPSA %: serum free PSA %.

| | | | AUC |
|---|---|---|---|
| V1 | V2 | Glycan_structure | (1 × CV) |
| Comp. 3 | Comp. 7 | H4N3F1S2,61; H4N4F1S2,61 | 69.8 |
| Comp. 7 | Comp. Mono.anntenary | H4N4F1S2,61; Mono-anntenary | 69.5 |
| Comp. 3 | Comp. 8 | H4N3F1S2,61; H5N4F1S2,61 | 69.1 |
| Comp. 2 | Comp. 24 | H4N3S2,61; H4N5S2,31(SO3)1 | 69.1 |
| Comp. 9 | Comp. Mono.anntenary | H5N4F1S2,61; Mono-anntenary | 68.4 |
| Comp. 2 | Comp. a2.3.sialylation | H4N3S2,61; a2,3-sialylation | 68.3 |
| Comp. 2 | Comp. 18 | H4N3S2,61: H5N4F1S2,31S2,61 | 68 |
| Comp. 2 | Comp. 7 | H4N3S2,61; H4N4F1S2,61 | 67.6 |
| Comp. 2 | Comp. 9 | H4N3S2.61; H5N4F1S2,61 | 67.4 |
| Comp. 2 | Comp. a2.6.sialylation | H4N3S2,61; a2,6-siaylation | 67.3 |
| Comp. 3 | Comp. 18 | H4N3F1S2,61; H5N4F1S2,31S2,61 | 67.3 |
| Comp. 18 | Comp. Mono.anntenany | H5N4F1S2,31S2,61; Mono-anatenary | 67.2 |
| Comp. 18 | Comp. 20 | H5N4F1S2,31S2,61; H4N5F1S2,62 | 67.1 |
| Comp. 2 | Comp. 21 | H4N3S2,61; H4N5F1S2,31S2,61 | 66.8 |
| Comp. 2 | Comp. 11 | H4N3S2.61; H4N5F1S2,63 | 66.7 |
| Comp. 10 | Comp. Mono.anntenany | H5N4F1S2,31; Mono-anntenary | 66.6 |
| Comp. 5 | Comp. 21 | H3N4F1S2,61; H4N5F1S2,31S2,61 | 66.5 |
| Comp. a2.3.sialylation | Comp. Mono.anntenary | a2,3-sialylation; Mono-anntenary | 66.2 |
| Comp. 21 | Comp. Mono.anntenary | H4N5F1S2,31S2,61; Mono-anntenary | 66.2 |
| Comp. 4 | Comp. Mono.anntenary | H4N3F1S2,31; Mono-anntenary | 66 |
| fPSA.perc | fPSA.perc | fPSA.perc | 58.7 |

TABLE 8
Multivariate performance of multivariate 3-compound combinations ranked according their AUC
value for differentiation of aggressive from indolent PCa and BPH. fPSA %: serum free PSA %.

| | | | | AUC |
|---|---|---|---|---|
| V1 | V2 | V3 | Glycan_structure | (1 × CV) |
| Comp. 4 | Comp. 7 | Comp. Mono.antennary | H4N3F1S2.31; H4N4F1S2,61; Mono-antennary | 73.1 |
| Comp. 2 | Comp. 11 | Comp. 13 | H4N3S2,61; H4N5F1S2,61; H5N4S2.31S2,61 | 72.8 |
| Comp. 2 | Comp. 9 | Comp. 13 | H4N3S2,61; H5N4F1S2,61; H5N4S2,31S2,61 | 72.6 |
| Comp. 3 | Comp. 7 | Comp. 18 | H4N3F1S2,61; H4N4F1S2,61; H5N4F1S2,31S2.61 | 72.5 |
| Comp. 3 | Comp. 7 | Comp. 21 | H4N3F1S2,61; H4N4F1S2,61; H4N5F1S2,31S2,61 | 72.5 |
| Comp. 2 | Comp. 7 | Comp. 13 | H4N3S2,61; H4N4F1S2,61; H5N4S2,31S2,61 | 72.3 |
| Comp. 2 | Comp. 9 | Comp. 24 | H4N3S2.61; H5N4F1S2,61; H4N5S2,31(SO3)1 | 72.2 |
| Comp. 7 | Comp. a2.3.sialylation | Comp. Mono.antennary | H4N4F1S2,61; a2,3-sialylation; Mono-antennary | 72.1 |
| Comp. 7 | Comp. 21 | Comp. Mono.antennary | H4N4F1S2,61; H4N5F1S2,31S2,61; Mono-antennary | 72 |
| Comp. 9 | Comp. a2.3.sialylation | Comp. Mono.antennary | H5N4F1S2,61; a2,3-sialylation; Mono-antennary | 71.1 |
| Comp. 2 | Comp. 7 | Comp. 24 | H4N3S2.61; H4N4F1S2,61; H4N5S2,31(SO3)1 | 71 |
| Comp. 3 | Comp. 7 | Comp. 9 | H4N3F1S2,61; H4N4F1S2,61; H5N4F1S2,61 | 71 |
| Comp. 3 | Comp. 7 | Comp. Hybrid.glycans | H4N3F1S2,61; H4N4F1S2,61; Hybrid glycans | 70.8 |
| Comp. 9 | Comp. 18 | Comp. Mono.antennary | H5N4F1S2,61; H5N4F1S2,31S2.61; Mono-antennary | 70.8 |
| Comp. 11 | Comp. 18 | Comp. Mono.antennary | H4N5F1S2,61; H5N4F1S2,31S2,61; Mono-antennary | 70.7 |
| fPSA.perc | Comp. 3 | Comp. 7 | fPSA.perc; H4N3F1S2,61; H4N4F1S2,61 | 70.7 |
| Comp. 7 | Comp. 18 | Comp. Mono.antennary | H4N4F1S2,61; H5N4F1S2,31S2,61; Mono-antennary | 70.6 |
| Comp. 2 | Comp. 7 | Comp. 16 | H4N3S2,61; H4N4F11S2.61; H4N5S2,31S2,63 | 70.5 |
| Comp. 2 | Comp. 24 | Comp. 25 | H4N3S2,61; H4N5S2,31(SO3)1; H4N5F1S2,61(SO3)1 | 70.4 |
| Comp. 7 | Comp. 10 | Comp. Mono.antennary | H4N4F1S2,61; H5N4F1S2.31; Mono-antennary | 70.3 |
| fPSA.perc | fPSA.perc | fPSA.perc | fPSA.perc | 58.4 |

TABLE 9
Multivariate performance of multivariate 4-compound combinations ranked according their AUC
value for differentiation of aggressive from indolent PCa and BPH. fPSA %: serum free PSA %.

| | | | | | AUC |
|---|---|---|---|---|---|
| V1 | V2 | V3 | V4 | Glycan_structure | (1 × CV) |
| Comp. 2 | Comp. 9 | Comp. 13 | Comp. 22 | H4N3S2,61; H5N4F1S2,61; H5N4S2,31S2,61; | 75.1 |
| | | | | H3N6F1S2,62 | |
| Comp. 3 | Comp. 7 | Comp. 18 | Comp. 20 | H4N3F1S2,61; H4N4F1S2,61; H5N4F1S2,31S2,61; | 74.6 |
| | | | | H4N5F1S2,62 | |
| Comp. 3 | Comp. 7 | Comp. 11 | Comp. 18 | H4N3F1S2,61; H4N4F1S2,61; H4N5F1S2,61; | 74.6 |
| | | | | H5N4F1S2.31S2,61 | |
| Comp. 2 | Comp. 11 | Comp. 13 | Comp. 16 | H4N3S2,61; H4N5F1S2,61; H5N4S2,31S2,61; | 74.5 |
| | | | | H4N5S2,31S2,61 | |
| Comp. 2 | Comp. 9 | Comp. 13 | Comp. 20 | H4N3S2,61; H5N4F1S2,61; H5N4S2,31S2,61; | 74.5 |
| | | | | H4N5F1S2,62 | |
| Comp. 2 | Comp. 11 | Comp. 12 | Comp. 18 | H4N3S2,61; H4N5F1S2,61; H5N4S2,62; | 74.2 |
| | | | | H5N4F1S2,31S2,61 | |
| Comp. 2 | Comp. 9 | Comp. 11 | Comp. 13 | H4N3S2,61; H5N4F1S2,61; H4N5F1S2,61; | 74.2 |
| | | | | H5N4S2,31S2.61 | |
| Comp. 2 | Comp. 7 | Comp. 11 | Comp. 13 | H4N3S2,61; H4N4F1S2,61; H4N5F1S2,61; | 73.9 |
| | | | | H5N4S2.31S2,61 | |
| Comp. 2 | Comp. 7 | Comp. 15 | Comp. 18 | H4N3S2,61; H4N4F152.61; H4N5S2.62; | 73.7 |
| | | | | H5N4F1S2,31S2.61 | |
| fPSA.perc | Comp. 3 | Comp. 7 | Comp. 21 | fPSA.perc; H4N3F1S2,61; H4N4F1S2.61; | 73.6 |
| | | | | H4N5F1S2,31S2,61 | |
| Comp. 4 | Comp. 7 | Comp. 21 | Comp. Mono.anntenary | H4N3F1S2,31; H4N4F1S2,61; H4N5F1S2.31S2.61; | 73.5 |
| | | | | Mono-anntenary | |
| Comp. 7 | Comp. 20 | Comp. a2.3.sialvlation | Comp. Mono.anntenary | H4N4F1S2,61; H4N5F1S2,62; a2,3-sialylation; | 73.1 |
| | | | | Mono-anntenary | |
| Comp. 3 | Comp. 7 | Comp. 15 | Comp. 18 | H4N3F1S2,61; H4N4F1S2.61; H4N5S2,62; | 72.8 |
| | | | | H5N4F1S2,31S2,61 | |
| Comp. 4 | Comp. 7 | Comp. 15 | Comp. Mono.anntenary | H4N3F1S2,31; H4N4F1S2,61; H4N5S2.62; | 72.7 |
| | | | | Mono-anntenary | |
| Comp. 2 | Comp. 7 | Comp. 13 | Comp. 20 | H4N3S2,61; H4N4F1S2,61; H5N4S2,31S2,61; | 72.6 |
| | | | | H4N5F1S2,62 | |
| Comp. 2 | Comp. 7 | Comp. 12 | Comp. 21 | H4N3S2,61; H4N4F1S2.61; H5N4S2,62; | 72 |
| | | | | H4N5F1S2,31S2,61 | |
| Comp. 7 | Comp. 15 | Comp. 18 | Comp. Mono.anntenary | H4N4F1S2,61; H4N5S2,62; H5N4F1S2,31S2,61; | 72 |
| | | | | Mono-anntenary | |
| Comp. 2 | Comp. 7 | Comp. 15 | Comp. a2.3.sialylation | H4N3S2,61; H4N4F1S2.61; H4N5S2.62; | 71.2 |
| | | | | a2.3-sialylation | |
| Comp. 4 | Comp. 7 | Comp. 24 | Comp. Mono.anntenary | H4N3F1S2,31; H4N4F1S2,61; H4N5S2,31(SO3)1; | 70.8 |
| | | | | Mono-anntenary | |
| Comp. 2 | Comp. 9 | Comp. 24 | Comp. 25 | H4N3S2,61; H5N4F1S2,61; H4N5S2.31(SO3)1; | 70.4 |
| | | | | H4N5F1S2,61(SO3)1 | |
| fPSA.perc | fPSA.perc | fPSA.perc | fPSA.perc | fPSA.perc | 57.6 |

### 1.4.5 Results—Ratios Analysis

As the selected combinations of two markers were most frequently +/− or −/+combinations, and the coefficients in the multivariate model (where logistic regression with log-transformed markers was used) had comparable absolute value, the identity c log(M₁)−c log(M₂)=c log(M₁/M₂) indicates the utility of marker ratios.

In FIG. 6 strongest up- and down-regulated traits are shown.

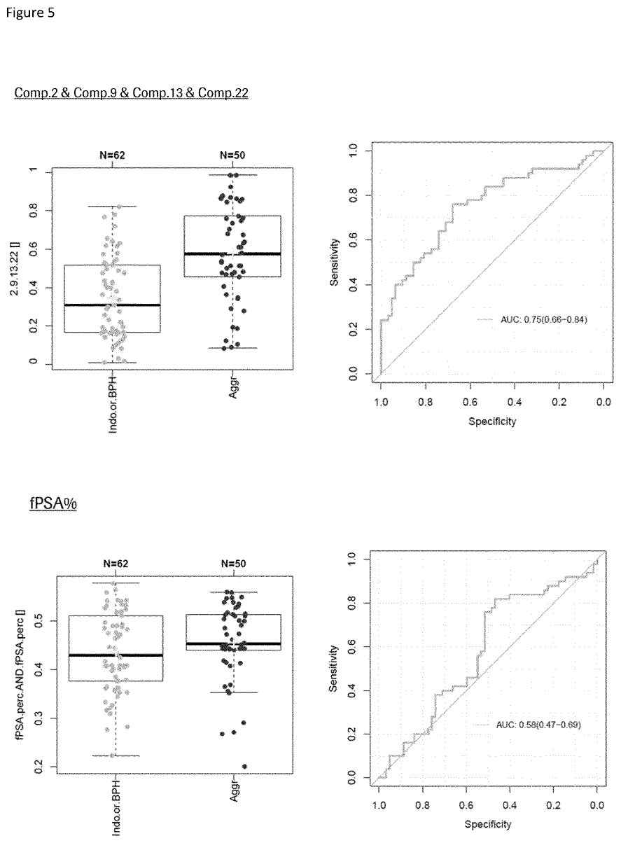

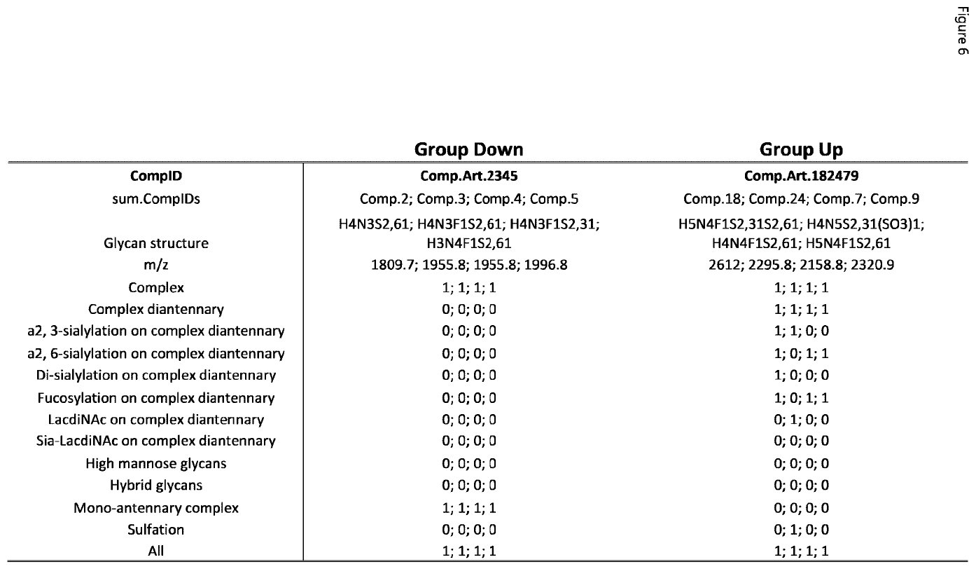

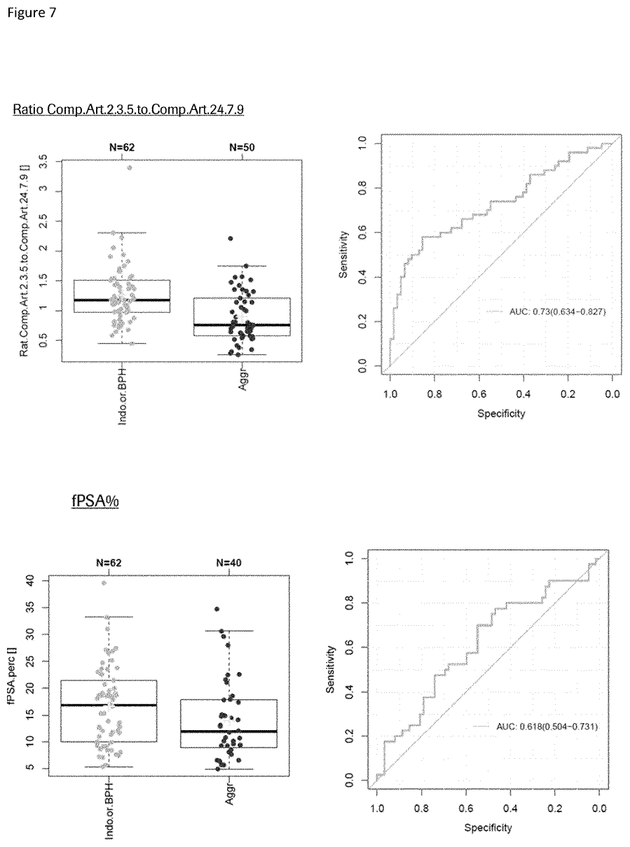

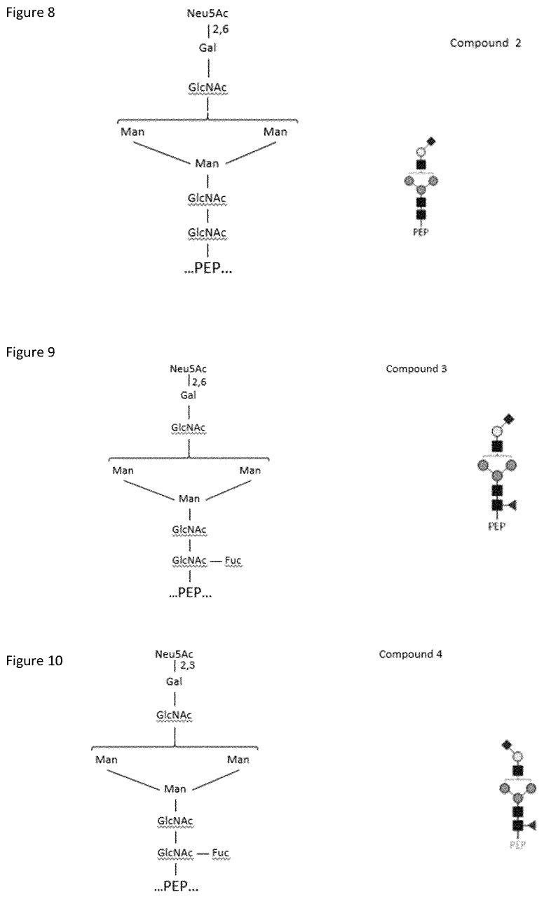

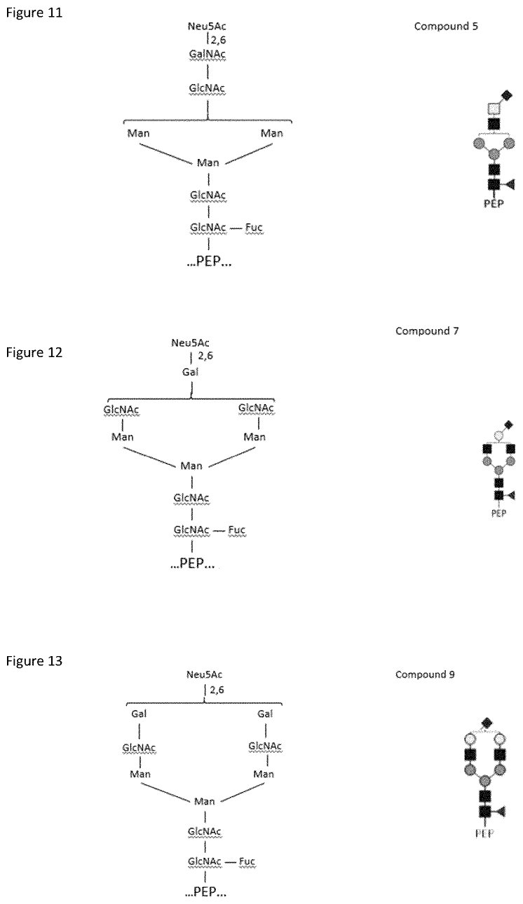

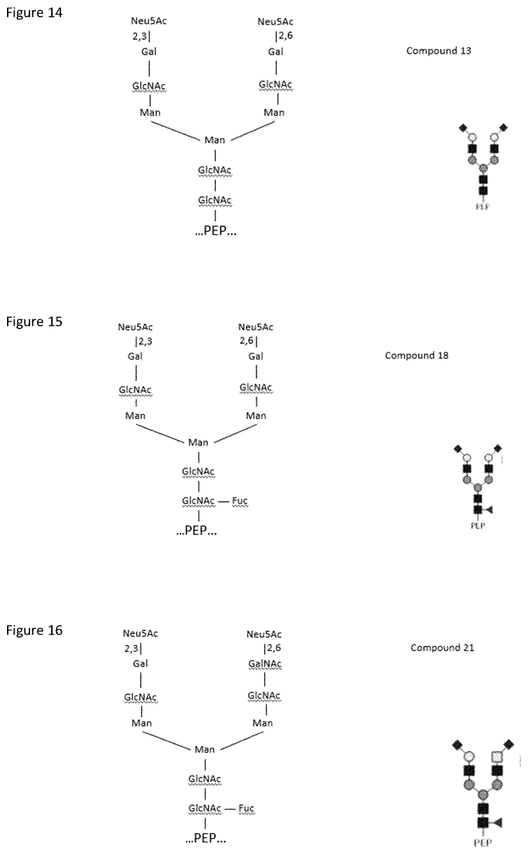

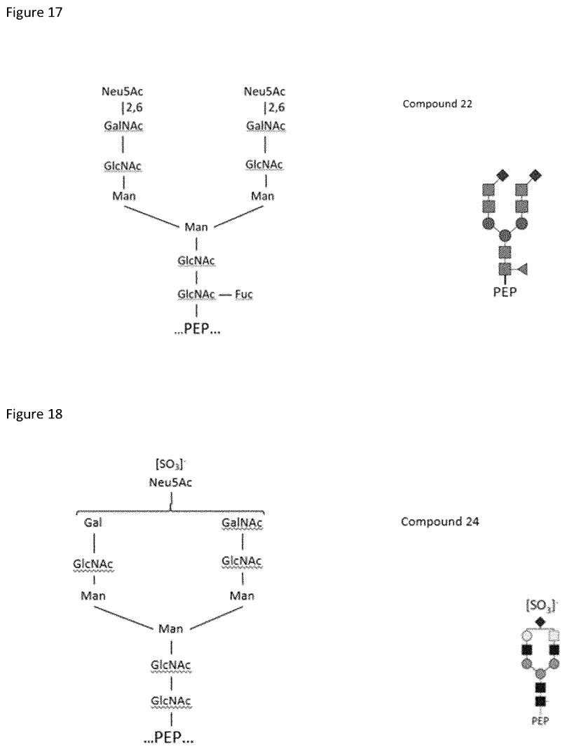

Biostatistical analysis demonstrated significant benefit to clinical performance through multivariate combination by including artificially combined traits (group up-regulated, group down-regulated) and calculation of their ratios (Table 10 and FIG. 7).

TABLE 10
Performance of ratios between major up- and down-regulated
compounds ranked according their AUC value for differentiation
of aggressive from indolent PCa and BPH. fPSA %: serum free
PSA %; Rat.: ratio; Comp. Art. = artificially combined
level of indicated compounds (combination is achieved by
building a sum of the levels of the indicated compounds).

| CompID | corr.p.value | AUG |
|---|---|---|
| Rat.Comp. Art.2.3.5.to.Comp. Art.24.7.9 | 4.62e−3 | 73 |
| Rat.Comp. Art.2.3.to.Comp. Art.7.9 | 4.62e−3 | 73 |
| Rat.Comp. Art.3.5.to.Comp. Art.24.7.9 | 4.62e−3 | 72.8 |
| Rat.Comp. Art.2.3.5.to.Comp. Art.7.9 | 4.62e−3 | 72.7 |
| Rat.Comp. Art.2.3.to.Comp. 7 | 4.62e−3 | 72.7 |
| Rat.Comp. Art.3.5.to.Comp. Art.7.9 | 4.62e−3 | 72.5 |
| Rat.Comp. Art.2.3.to.Comp. 9 | 4.62e−3 | 72.4 |
| Rat.Comp. Art.3.5.to.Comp. Art.24.9 | 4.62e−3 | 72.4 |
| Rat.Comp. Art.2.3.to.Comp. Art.24.7.9 | 4.62e−3 | 72.3 |
| Rat.Comp. Art.7.9.to.Comp. 3 | 4.62e−3 | 72.3 |
| Rat.Comp. 3.to.Comp. 9 | 4.626−3 | 72.2 |
| Rat.Comp. Art.2.3.5.to.Comp. 7 | 4.62e−3 | 72.2 |
| Rat.Comp. Art.2.3.5.to.Comp. 9 | 4.62e−3 | 72.2 |
| Rat.Comp. Art.3.5.to.Comp. 9 | 4.62e−3 | 72.1 |
| Rat.Comp. Art.24.7.9.to.Comp. 3 | 4.88e−3 | 71.9 |
| Rat.Comp. Art.2.3.5.to.Comp. Art.24.9 | 5.03e−3 | 71.5 |
| Rat.Comp. Art.2.3.to.Comp. Art.18.24.7.9 | 5.03e−3 | 71.5 |
| Rat.Comp. Art.2.3.to.Comp. Art.24.9 | 5.03e−3 | 71.5 |
| Rat.Comp. Art.2.3.5.to.Comp. Art.18.24.7.9 | 5.03e−3 | 71.4 |
| Rat.Comp. Art.2.3.5.to.Comp. Art.18.7.9 | 5.03e−3 | 71.4 |
| fPSA.perc | 1.04e−1 | 61.8 |

Interestingly, all major down-regulated structures, Comp. 2, 3, 4, 5, represent complex, mono-antennary N-glycans, while up-regulated group, Comp, 7, 9, 18 and 24, contains specifically complex di-antennary, sialylated and fucosylated N-glycans. Therefore, it might be concluded that the ratio between mono- and di-antennary PSA glycoforms is a novel marker (feature) for improved differentiation of the aggressive PCa from its indolent form and benign controls.

### 1.5 Conclusions

CE-MS-based glyco-analysis of the urinary PSA glyoforms revealed several novel structures and structure combinations with the superior clinical performance for differentiating aggressive from indolent prostate carcinoma as compared to serum fPSA %:
In univariate setting: three glycoforms with larger AUC than serum fPSA %
In multivariate 2-marker setting: approx. 6% improvement to univariate model
In multivariate 3-marker setting: approx. 10% improvement to univariate model
In multivariate 3-marker setting: approx. 12% improvement to univariate model, approx. 5-10% improvement to Beckmann prostate health index (PHI) (serum)
Significant benefit to clinical performance through multivariate combination of artificially combined traits (Group Up, Group Down) and ratios, approx. 10% improvement to univariate model, approx. 5-10% improvement to Beckmann PHI (serum).

1. A method to aid in determining whether or not a subject has an aggressive form of prostate cancer, comprising:
(a) determining the level of at least one mono-antennary PSA glycoform and the level of at least one di-antennary PSA glycoform in a biofluid sample from the subject;
(b) calculating an aggressiveness score for the subject based on the levels determined in (a);
(c) comparing the subject's aggressiveness score in (b) to a reference value for said aggressiveness score; and
(d) using the comparison in (c) to aid in determining whether the subject has an aggressive form of prostate cancer or not.

2. A method according to claim 1, wherein step (a) comprises:
(i) isolating PSA protein from a biofluid sample from the subject;
(ii) treating the isolated PSA protein with a protease, such as trypsin or Arg-C (clostripain);
(iii) determining the level of each of two or more N₆₉ comprising glycopeptide forms in the sample, wherein at least one of the N₆₉ comprising glycopeptide forms is a mono-antennary glycoform and at least one of the N₆₉ comprising glycopeptide forms is a di-antennary glycoform.

3. The method according to claim 1, wherein the at least one mono-antennary PSA glycoform is selected from the group consisting of: H4N3S_{2,6}1; H4N3F1S_{2,6}1; H4N3F1S_{2,3}1; and H3N4F1S_{2,6}1.

4. The method according to claim 1, wherein the at least one di-antennary PSA glycoform is selected from the group consisting of: H4N4F1S_{2,6}1; H5N4F1S_{2,6}1; H5N4F1S_{2,3}1; H4N5F1S_{2,6}1; H5N4S_{2,3}1S_{2,6}1; H4N5S_{2,3}1S_{2,6}1; H4N5F1S_{2,3}1S_{2,6}1; H4N5S_{2,3}1(SO₃)1; and H4N5F1S_{2,6}1(SO₃)1.

5. The method according to claim 1, wherein the aggressiveness score comprises or consists of a ratio of the level of the at least one di-antennary PSA glycoform from the subject sample over the at least one mono-antennary PSA glycoform or a vice versa ratio.

6. The method according to claim 1, wherein the PSA glycoforms used or determined in the methods of the invention comprise PSA glycoforms selected from a group consisting of:
(i) H4N3F1S_{2,6}1 and H4N4F1S_{2,6}1;
(ii) H4N3F1S_{2,6}1 and H5N4F1S_{2,6}1;
(iii) H4N3S_{2,6}1 and H4N5S_{2,3}1(SO₃)1;
(iv) H4N3S_{2,6}1 and H5N4F1S_{2,3}1S_{2,6}1;
(v) H4N3S_{2,6}1 and H4N4F1S_{2,6}1;
(vi) H4N3F1S_{2,6}1 and H5N4F1S_{2,3}1S_{2,6}1;
(vii) H4N3S_{2,6}1 and H4N5F1S_{2,3}1S_{2,6}1;
(viii) H4N3S_{2,6}1 and H4N5F1S_{2,6}1; and
(ix) H3N4F1S_{2,6}1 and H4N5F1S_{2,3}1S_{2,6}1.

7. The method according to claim 1, wherein the PSA glycoforms used or determined in the methods of the invention comprise PSA glycoforms selected from a group consisting of:
a. H4N4F1S_{2,6}1 and a combination of mono-antennary glycoforms;
b. H4N3S_{2,6}1 and a combination of mono-antennary glycoforms;
c. H4N3S_{2,6}1 and a combination of a2.3.sialylation glycoforms;
d. H4N3S_{2,6}1 and a combination of a2.6.sialylation glycoforms;
e. H5N4F1S_{2,3}1S_{2,6}1 and a combination of mono-antennary glycoforms;
f. H5N4F1S_{2,3}1 and a combination of mono-antennary glycoforms;
g. a combination of a2.3.sialylation glycoforms and a combination of mono- antennary glycoforms;
h. H4N5F1S_{2,3}1S_{2,6}1 and a combination of mono-antennary glycoforms; and
i. H4N3F1S_{2,3}1 and a combination of mono-antennary glycoforms.

8. The method according to claim 1, wherein the subject is a mammal, such as a male human, and/or wherein the biofluid sample is selected from: blood, serum, plasma, seminal fluid, prostatic fluid and urine, such as a digital rectal examination (DRE) urine sample.

9. The method according to claim 1, wherein the subject is preselected based on having prostate-gland enlargement or based on the level of total serum PSA, optionally wherein the level of total serum PSA in the subject is between about 2-10 ng/ml.

10. The method according to claim 1, wherein the PSA protein in the subject's biofluid sample is isolated from the sample, optionally by use of a capture binding partner, such as an anti-PSA antibody.

11. The method according to claim 10, wherein the isolated PSA protein is treated with a protease to produce N₆₉ comprising glycopeptides.

12. The method according to claim 1, wherein the level of each measured PSA glycoform is determined by mass spectrometry, such as CE-ESI-MS or LC-ESI-MS(/MS), or MALDI-TOF MS.

13. The method according to claim 1, wherein the reference value for the aggressiveness score has been determined from samples of at least one reference population comprising aggressive PCa subjects and subjects that do not have aggressive PCa, such as patients with prostatitis, BPH or indolent PCa, optionally wherein said disease status is known.

14. A kit for use in a method according to claim 1, comprising a scoring system with PSA glycoform reference values or ratios indicative of aggressive PCa, and optionally (i) instructions for use, and/or (ii) a PSA binding partner, such as a monoclonal antibody, optionally wherein the PSA binding partner is immobilised onto a solid surface, such as a bead.

15. A computer-implemented method to aid in determining whether a subject has an aggressive form of prostate cancer or not, comprising the steps of:
(a) receiving a value for the level of a first mono-antennary PSA glycoform in a biofluid sample of the subject;
(b) receiving a value for the level of a first di-antennary PSA glycoform in the biofluid sample of the subject;
(c) calculating an aggressiveness score for the subject based on the levels received in (a) and (b);
(d) comparing the subject's aggressiveness score in (c) to a reference value for said aggressiveness score; and
(e) using the comparison in (d) to aid in determining whether the subject has an aggressive form of prostate cancer or not.
